(19) United States
(12) Patent Application Publication
(10) Pub. No.: US 2013/0122149 A1
(43) Pub. Date: May 16, 2013

(54) Polypeptides Having Glucose Dehydrogenase Activity and Polynucleotides Encoding Same

(71) Applicant: NOVOZYMES A/S, Bagsvaerd (DK)

(72) Inventors: Miguel Duarte Toscano, Copenhagen (DK); Henrik Ostdal, Virum (DK); Rune Gerner Moller, Fredenborg (DK); Roland Ludwig, Vienna (AT); Christoph Sygmund, Klosterneuburg (AT)

(73) Assignee: NOVOZYMES A/S, Bagsvaerd (DK)

(21) Appl. No.: 13/742,740

(22) Filed: Jan. 16, 2013

Related U.S. Application Data
(63) Continuation of application No. 13/249,363, filed on Sep. 30, 2011, now abandoned.

(51) Int. Cl. C12N 9/00

(52) U.S. Cl. CPC C12N 9/0006
USPC 426/61; 435/190; 536/23.2; 435/320.1; 435/252.31; 435/252.3; 435/252.35; 435/252.34; 435/325; 435/348; 435/419; 435/254.11; 435/254.2; 435/254.22; 435/254.23; 435/254.21; 435/254.3; 435/254.7; 435/254.4; 435/254.5; 435/254.6; 435/252.33

(57) ABSTRACT

The present invention relates to isolated polypeptides having glucose dehydrogenase activity, and polynucleotides encoding the polypeptides. The invention also relates to nucleic acid constructs, vectors, and host cells comprising the polynucleotides as well as methods of producing and using the polypeptides.

### CROSS-REFERENCE TO RELATED APPLICATIONS

This application is a continuation of U.S. application Ser. No. 13/249,363 filed Sep. 30, 2011 (pending), the contents of which are fully incorporated herein by reference.

### REFERENCE TO A SEQUENCE LISTING

This application contains a Sequence Listing in computer readable form, which is incorporated herein by reference.

### BACKGROUND OF THE INVENTION

1. Field of the Invention

The present invention relates to polypeptides having glucose dehydrogenase activity, and polynucleotides encoding the polypeptides. The invention also relates to nucleic acid constructs, vectors, and host cells comprising the polynucleotides as well as methods of producing and using the polypeptides.

2. Description of the Related Art

The glucose dehydrogenase of the present invention has been disclosed in Master Thesis by Miriam Klausberger, titled Cloning, expression and characterisation of a novel FAD-dependent glucose dehydrogenase from University of Natural Recourses and Applied Life Sciences, Vienna, Austria and published online 4 Oct. 2010, in conference abstract (62^{nd} of the International Society of Electrochemistry Annual Meeting) by M. Nadeem Zafara et al. titled Electron transfer studies with different sugar oxidizing enzymes and osmium polymers to improve the current density and published online August 2011, and in article by C. Sygmund et al. titled Reduction of quinones and phenoxy radicals by extracellular glucose dehydrogenase from Glomerella cingulata suggests a role in plant pathogenicity, and published online 8 Sep. 2011 (Microbiology Papers, in press); all of which are hereby incorporated by reference.

The glucose dehydrogenase of the present invention is approximately 57% identical to a glucose dehydrogenase from Penicillium lilacinoechinulatum disclosed in US2007/0105174.

The use of glucose dehydrogenase for dough conditioning has been proposed in WO9957986.

Dough “conditioners” are well known in the baking industry. The addition of conditioners or to bread dough has resulted in improved machinability of the dough and improved texture, volume, flavor, and freshness (anti-staling) of the bread. Nonspecific oxidants, such as iodates, peroxides, ascorbic acid, potassium bromate and azodicarbonamide have a gluten strengthening effect. However, the use of several of the currently available chemical oxidizing agents has been met with consumer resistance or is not permitted by regulatory agencies.

The use of enzymes as dough conditioners has been considered as an alternative to chemical conditioners. A number of enzymes are used as dough and/or bread improving agents, in particular, enzymes that act on components present in large amounts in the dough. Examples of such enzymes are lipases, amylases, proteases, glucose oxidases, cellulases, and xylanases.

### SUMMARY OF THE INVENTION

The inventors of the present application have isolated a new glucose dehydrogenase from the ascomycete fungus Glomerella cingulata (anamorph Colletotrichum gloeosporoides).

Accordingly, the present invention provides polypeptides having glucose dehydrogenase activity and polynucleotides encoding the polypeptides. The glucose dehydrogenase of the present invention is particularly suited for use in the preparation of a baked product due to the low thermal stability. This ensures that the enzyme is deactivated in the heat treatment during the preparation of a baked product. The final baked product will thus only comprise the denatured and inactive enzyme protein.

The present invention relates to isolated polypeptides having glucose dehydrogenase activity selected from the group consisting of:

(a) a polypeptide having at least 60% sequence identity to the mature polypeptide of SEQ ID NO: 2;

(b) a polypeptide encoded by a polynucleotide that hybridizes under medium high stringency conditions with (i) the mature polypeptide coding sequence of SEQ ID NO: 1 or SEQ ID NO: 3, (ii) the full-length complement of (i);

(c) a polypeptide encoded by a polynucleotide having at least 60% sequence identity to the mature polypeptide coding sequence of SEQ ID NO: 1 or SEQ ID NO: 3;

(d) a variant of the mature polypeptide of SEQ ID NO: 2 comprising a substitution, deletion, and/or insertion at one or more (e.g., several) positions; and
(e) a fragment of the polypeptide of (a), (b), (c), or (d) that has glucose dehydrogenase activity.

The present invention also relates to isolated polynucleotides encoding the polypeptides of the present invention; nucleic acid constructs; recombinant expression vectors; recombinant host cells comprising the polynucleotides; and methods of producing the polypeptides.

The present invention also relates to methods of using the polypeptides for preparing a dough or a baked product.

### Definitions

Glucose dehydrogenase: The term “glucose dehydrogenase” means a polypeptide having an activity (EC 1.1.99.10) that catalyzes the reaction D-glucose+acceptor=D-glucono-1,5-lactone+reduced acceptor. The enzyme from Glomerella cingulata is a monomeric glycosylated polypeptide and has one non-covalently bound flavin adenine dinucleotide (FAD) molecule acting as the acceptor.

For purposes of the present invention, one unit of glucose dehydrogenase activity is defined as the amount of enzyme necessary for the reduction of 1 pmol glucose per min under the assay conditions described in Example 3. In one aspect, the polypeptides of the present invention have at least 20%, e.g., at least 40%, at least 50%, at least 60%, at least 70%, at least 80%, at least 90%, at least 95%, or at least 100% of the glucose dehydrogenase activity of the mature polypeptide of SEQ ID NO: 2.

Allelic variant: The term “allelic variant” means any of two or more alternative forms of a gene occupying the same chromosomal locus. Allelic variation arises naturally through mutation, and may result in polymorphism within populations. Gene mutations can be silent (no change in the encoded polypeptide) or may encode polypeptides having altered amino acid sequences. An allelic variant of a polypeptide is a polypeptide encoded by an allelic variant of a gene.

cDNA: The term “cDNA” means a DNA molecule that can be prepared by reverse transcription from a mature, spliced, mRNA molecule obtained from a eukaryotic or prokaryotic cell. cDNA lacks intron sequences that may be present in the corresponding genomic DNA. The initial, primary RNA transcript is a precursor to mRNA that is processed through a series of steps, including splicing, before appearing as mature spliced mRNA.

Coding sequence: The term “coding sequence” means a polynucleotide, which directly specifies the amino acid sequence of a polypeptide. The boundaries of the coding sequence are generally determined by an open reading frame, which begins with a start codon such as ATG, GTG, or TTG and ends with a stop codon such as TAA, TAG, or TGA. The coding sequence may be a genomic DNA, cDNA, synthetic DNA, or a combination thereof.

Control sequences: The term “control sequences” means nucleic acid sequences necessary for expression of a polynucleotide encoding a mature polypeptide of the present invention. Each control sequence may be native (i.e., from the same gene) or foreign (i.e., from a different gene) to the polynucleotide encoding the polypeptide or native or foreign to each other. Such control sequences include, but are not limited to, a leader, polyadenylation sequence, propeptide sequence, promoter, signal peptide sequence, and transcription terminator. At a minimum, the control sequences include a promoter, and transcriptional and translational stop signals. The control sequences may be provided with linkers for the purpose of introducing specific restriction sites facilitating ligation of the control sequences with the coding region of the polynucleotide encoding a polypeptide.

Expression: The term “expression” includes any step involved in the production of a polypeptide including, but not limited to, transcription, post-transcriptional modification, translation, post-translational modification, and secretion.

Expression vector: The term “expression vector” means a linear or circular DNA molecule that comprises a polynucleotide encoding a polypeptide and is operably linked to control sequences that provide for its expression.

Fragment: The term “fragment” means a polypeptide having one or more (e.g., several) amino acids absent from the amino and/or carboxyl terminus of a mature polypeptide; wherein the fragment has glucose dehydrogenase activity. In one aspect, a fragment contains at least 580 amino acid residues, at least 570 amino acid residues, or at least 560 amino acid residues.

Host cell: The term “host cell” means any cell type that is susceptible to transformation, transfection, transduction, or the like with a nucleic acid construct or expression vector comprising a polynucleotide of the present invention. The term “host cell” encompasses any progeny of a parent cell that is not identical to the parent cell due to mutations that occur during replication.

Isolated: The term “isolated” means a substance in a form or environment that does not occur in nature. Non-limiting examples of isolated substances include (1) any non-naturally occurring substance, (2) any substance including, but not limited to, any enzyme, variant, nucleic acid, protein, peptide or cofactor, that is at least partially removed from one or more or all of the naturally occurring constituents with which it is associated in nature; (3) any substance modified by the hand of man relative to that substance found in nature; or (4) any substance modified by increasing the amount of the substance relative to other components with which it is naturally associated (e.g., multiple copies of a gene encoding the substance; use of a stronger promoter than the promoter naturally associated with the gene encoding the substance). An isolated substance may be present in a fermentation broth sample.

Mature polypeptide: The term “mature polypeptide” means a polypeptide in its final form following translation and any post-translational modifications, such as N-terminal processing, C-terminal truncation, glycosylation, phosphorylation, etc. In one aspect, the mature polypeptide is amino acids 1 to 585 of SEQ ID NO: 2 based on the programs Compute pl/Mw and SignalP hosted by the Expasy Proteomics server (www.expasy.ch) that predict amino acids −1 to −14 of SEQ ID NO: 2 are a signal peptide. It is known in the art that a host cell may produce a mixture of two of more different mature polypeptides (i.e., with a different C-terminal and/or N-terminal amino acid) expressed by the same polynucleotide.

Mature polypeptide coding sequence: The term “mature polypeptide coding sequence” means a polynucleotide that encodes a mature polypeptide having glucose dehydrogenase activity. In one aspect, the mature polypeptide coding sequence is nucleotides 43 to 1800 of SEQ ID NO: 1 or of SEQ ID NO: 3 based on the programs Compute pl/Mw and SignalP hosted by the Expasy Proteomics server (www.expasy.ch) that predict nucleotides 1 to 42 of SEQ ID NO: 1 or of SEQ ID NO: 3 to encode a signal peptide. SEQ ID NO: 1 is the native mature polypeptide coding sequence while SEQ ID NO: 3 is a synthetic mature polypeptide coding sequence codon optimized for recombinant expression of the glucose dehydrogenase in Aspergillus oryzae.

Nucleic acid construct: The term “nucleic acid construct” means a nucleic acid molecule, either single- or double-stranded, which is isolated from a naturally occurring gene or is modified to contain segments of nucleic acids in a manner that would not otherwise exist in nature or which is synthetic, which comprises one or more control sequences.

Operably linked: The term “operably linked” means a configuration in which a control sequence is placed at an appropriate position relative to the coding sequence of a polynucleotide such that the control sequence directs expression of the coding sequence.

Sequence identity: The relatedness between two amino acid sequences or between two nucleotide sequences is described by the parameter “sequence identity”.

For purposes of the present invention, the sequence identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later. The parameters used are gap open penalty of 10, gap extension penalty of 0.5, and the EBLOSUM62 (EMBOSS version of BLOSUM62) substitution matrix. The output of Needle labeled “longest identity” (obtained using the -nobrief option) is used as the percent identity and is calculated as follows:

(Identical Residues×100)/(Length of Alignment−Total Number of Gaps in Alignment)

For purposes of the present invention, the sequence identity between two deoxyribonucleotide sequences is determined using the Needleman-Wunsch algorithm

(Needleman and Wunsch, 1970, supra) as implemented in the Needle program of the EMBOSS package (EMBOSS: The European Molecular Biology Open Software Suite, Rice et al., 2000, supra), preferably version 5.0.0 or later. The parameters used are gap open penalty of 10, gap extension penalty of 0.5, and the EDNAFULL (EMBOSS version of NCBI NUC4.4) substitution matrix. The output of Needle labeled “longest identity” (obtained using the -nobrief option) is used as the percent identity and is calculated as follows:

(Identical Deoxyribonucleotides×100)/(Length of Alignment−Total Number of Gaps in Alignment).

Stringency conditions: The term “very low stringency conditions” means for probes of at least 100 nucleotides in length, prehybridization and hybridization at 42° C. in 5×SSPE, 0.3% SDS, 200 micrograms/ml sheared and denatured salmon sperm DNA, and 25% formamide, following standard Southern blotting procedures for 12 to 24 hours. The carrier material is finally washed three times each for 15 minutes using 2×SSC, 0.2% SDS at 45° C.

The term “low stringency conditions” means for probes of at least 100 nucleotides in length, prehybridization and hybridization at 42° C. in 5×SSPE, 0.3% SDS, 200 micrograms/ml sheared and denatured salmon sperm DNA, and 25% formamide, following standard Southern blotting procedures for 12 to 24 hours. The carrier material is finally washed three times each for 15 minutes using 2×SSC, 0.2% SDS at 50° C.

The term “medium stringency conditions” means for probes of at least 100 nucleotides in length, prehybridization and hybridization at 42° C. in 5×SSPE, 0.3% SDS, 200 micrograms/ml sheared and denatured salmon sperm DNA, and 35% formamide, following standard Southern blotting procedures for 12 to 24 hours. The carrier material is finally washed three times each for 15 minutes using 2×SSC, 0.2% SDS at 55° C.

The term “medium-high stringency conditions” means for probes of at least 100 nucleotides in length, prehybridization and hybridization at 42° C. in 5×SSPE, 0.3% SDS, 200 micrograms/ml sheared and denatured salmon sperm DNA, and either 35% formamide, following standard Southern blotting procedures for 12 to 24 hours. The carrier material is finally washed three times each for 15 minutes using 2×SSC, 0.2% SDS at 60° C.

The term “high stringency conditions” means for probes of at least 100 nucleotides in length, prehybridization and hybridization at 42° C. in 5×SSPE, 0.3% SDS, 200 micrograms/ml sheared and denatured salmon sperm DNA, and 50% formamide, following standard Southern blotting procedures for 12 to 24 hours. The carrier material is finally washed three times each for 15 minutes using 2×SSC, 0.2% SDS at 65° C.

The term “very high stringency conditions” means for probes of at least 100 nucleotides in length, prehybridization and hybridization at 42° C. in 5×SSPE, 0.3% SDS, 200 micrograms/ml sheared and denatured salmon sperm DNA, and 50% formamide, following standard Southern blotting procedures for 12 to 24 hours. The carrier material is finally washed three times each for 15 minutes using 2×SSC, 0.2% SDS at 70° C.

Subsequence: The term “subsequence” means a polynucleotide having one or more (e.g., several) nucleotides absent from the 5′ and/or 3′ end of a mature polypeptide coding sequence; wherein the subsequence encodes a fragment having glucose dehydrogenase activity. In one aspect, a subsequence contains at least 1740 nucleotides (e.g., nucleotides 1 to 1740 of SEQ ID NO: 1 or SEQ ID NO: 3), at least 1710 nucleotides (e.g., nucleotides 1 to 1710 of SEQ ID NO: 1 or SEQ ID NO: 3), or at least 1680 nucleotides (e.g., nucleotides 1 to 1680 of SEQ ID NO: 1 or SEQ ID NO: 3).

Variant: The term “variant” means a polypeptide having glucose dehydrogenase activity comprising an alteration, i.e., a substitution, insertion, and/or deletion, at one or more (e.g., several) positions, such as at 10 positions or less, at 9 positions or less, at 8 positions or less, at 7 positions or less, at 6 positions or less, at 5 positions or less, at 4 positions or less, at 3 positions or less, at 2 positions or less, such as at one position. A substitution means replacement of the amino acid occupying a position with a different amino acid; a deletion means removal of the amino acid occupying a position; and an insertion means adding an amino acid adjacent to and immediately following the amino acid occupying a position.

The phrase “incorporating into the dough” is defined herein as adding the glucose dehydrogenase(s) to the dough, any ingredient from which the dough is to be made, and/or any mixture of dough ingredients from which the dough is to be made. In other words, the glucose dehydrogenase(s) may be added in any step of the dough preparation and may be added in one, two, or more steps.

The term “effective amount” is defined herein as an amount of glucose dehydrogenase(s) that is sufficient for providing a measurable effect on at least one property of interest of the dough and/or baked product.

The term “improved property” is defined herein as any property of a dough and/or a product obtained from the dough, particularly a baked product, which is improved by the action of a glucose dehydrogenase relative to a dough or product in which a glucose dehydrogenase is not incorporated. The improved property may include, but is not limited to, increased strength of the dough, increased elasticity of the dough, increased stability of the dough, reduced stickiness of the dough, improved extensibility of the dough, improved machinability of the dough, increased volume of the baked product, improved crumb structure of the baked product, improved crust of the baked product, improved bloom of the baked product, improved softness of the baked product, improved flavor of the baked product, and/or improved antistaling of the baked product.

The term “increased strength of the dough” is defined herein as the property of a dough that has generally more elastic properties and/or requires more work input to mould and shape the dough.

The term “increased elasticity of the dough” is defined herein as the property of a dough which has a higher tendency to regain its original shape after being subjected to a certain physical strain.

The term “increased stability of the dough” is defined herein as the property of a dough that is less susceptible to mechanical abuse thus better maintaining its shape and volume.

The term “reduced stickiness of the dough” is defined herein as the property of a dough that has less tendency to adhere to surfaces, e.g., in the dough production machinery, and is either evaluated empirically by the skilled test baker or measured by the use of a texture analyzer (e.g., TAXT2) known in the art.

The term “improved extensibility of the dough” is defined herein as the property of a dough that can be subjected to increased strain or stretching without rupture.

The term “improved machinability of the dough” is defined herein as the property of a dough that is generally less sticky and/or more firm and/or more elastic.

The term “increased volume of the baked product” is measured as the specific volume of a given loaf of bread (volume/weight) determined typically by the traditional rape seed displacement method.

The term “improved crumb structure of the baked product” is defined herein as the property of a baked product with finer and/or thinner cell walls in the crumb and/or more uniform/homogenous distribution of cells in the crumb and is usually evaluated empirically by the skilled test baker.

The term “improved crust of the baked product” is usually evaluated empirically by the skilled test baker.

The term “bloom of the baked product” is the opening up of the scoring cuts during baking. Increased bloom is usually evaluated empirically by the skilled test baker or measured in mm.

The term “improved softness of the baked product” is the opposite of “firmness” and is defined herein as the property of a baked product that is more easily compressed and is evaluated either empirically by the skilled test baker or measured by the use of a texture analyzer (e.g., TAXT2) known in the art.

The term “improved flavor of the baked product” is evaluated by a trained test panel.

The term “improved antistaling of the baked product” is defined herein as the properties of a baked product that have a reduced rate of deterioration of quality parameters, e.g., softness and/or elasticity, during storage.

The term “dough” is defined herein as a mixture of flour and other ingredients firm enough to knead or roll. The dough may be fresh, frozen, pre-bared, or pre-baked. The preparation of frozen dough is described by Kulp and Lorenz in Frozen and Refrigerated Doughs and Batters.

The term “baked product” is defined herein as any product, either of a soft or a crisp character, prepared from heat treatment of a dough. Examples of baked products, whether of a white, light or dark type, which may be advantageously produced by the present invention are bread (in particular white, whole-meal or rye bread), typically in the form of loaves or rolls, French baguette-type bread, pasta, pita bread, tortillas, tacos, cakes, pancakes, biscuits, cookies, pie crusts, steamed bread, and crisp bread, and the like.

The term “heat treatment of a dough” include baking, steaming, and frying of a dough.

### DETAILED DESCRIPTION OF THE INVENTION

### Polypeptides Having Glucose Dehydrogenase Activity

In an embodiment, the present invention relates to isolated polypeptides having a sequence identity to the mature polypeptide of SEQ ID NO: 2 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%, which have glucose dehydrogenase activity. In one aspect, the polypeptides differ by no more than 10 amino acids, e.g., 1, 2, 3, 4, 5, 6, 7, 8, or 9, from the mature polypeptide of SEQ ID NO: 2.

A polypeptide of the present invention preferably comprises or consists of the amino acid sequence of SEQ ID NO: 2 or an allelic variant thereof; or is a fragment thereof having glucose dehydrogenase activity. In another aspect, the polypeptide comprises or consists of the mature polypeptide of SEQ ID NO: 2. In another aspect, the polypeptide comprises or consists of amino acids 1 to 586 of SEQ ID NO: 2.

In an embodiment, the present invention relates to isolated polypeptides encoded by a polynucleotide having a sequence identity to the mature polypeptide coding sequence of SEQ ID NO: 1 or SEQ ID NO: 3 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%.

In another embodiment, the present invention relates to an isolated polypeptide having glucose dehydrogenase activity encoded by a polynucleotide that hybridizes under very low stringency conditions, low stringency conditions, medium stringency conditions, medium-high stringency conditions, high stringency conditions, or very high stringency conditions with (i) the mature polypeptide coding sequence of SEQ ID NO: 1 or SEQ ID NO: 3, or (ii) the full-length complement of (i) (Sambrook et al., 1989, Molecular Cloning, A Laboratory Manual, 2d edition, Cold Spring Harbor, N.Y.).

The polynucleotide of SEQ ID NO: 1 or SEQ ID NO: 3 or a subsequence thereof, as well as the polypeptide of SEQ ID NO: 2 or a fragment thereof, may be used to design nucleic acid probes to identify and clone DNA encoding polypeptides having glucose dehydrogenase activity from strains of different genera or species according to methods well known in the art. In particular, such probes can be used for hybridization with the genomic DNA or cDNA of a cell of interest, following standard Southern blotting procedures, in order to identify and isolate the corresponding gene therein. Such probes can be considerably shorter than the entire sequence, but should be at least 15, e.g., at least 25, at least 35, or at least 70 nucleotides in length. Preferably, the nucleic acid probe is at least 100 nucleotides in length, e.g., at least 200 nucleotides, at least 300 nucleotides, at least 400 nucleotides, at least 500 nucleotides, at least 600 nucleotides, at least 700 nucleotides, at least 800 nucleotides, or at least 900 nucleotides in length. Both DNA and RNA probes can be used. The probes are typically labeled for detecting the corresponding gene (for example, with ³²P, ³H, ³⁵S, biotin, or avidin). Such probes are encompassed by the present invention.

A genomic DNA or cDNA library prepared from such other strains may be screened for DNA that hybridizes with the probes described above and encodes a polypeptide having glucose dehydrogenase activity. Genomic or other DNA from such other strains may be separated by agarose or polyacrylamide gel electrophoresis, or other separation techniques. DNA from the libraries or the separated DNA may be transferred to and immobilized on nitrocellulose or other suitable carrier material. In order to identify a clone or DNA that hybridizes with SEQ ID NO: 1 or SEQ ID NO: 3 or a subsequence thereof, the carrier material is used in a Southern blot.

For purposes of the present invention, hybridization indicates that the polynucleotide hybridizes to a labeled nucleic acid probe corresponding to (i) SEQ ID NO: 1 or SEQ ID NO: 3; (ii) the mature polypeptide coding sequence of SEQ ID NO: 1 or SEQ ID NO: 3; (iii) the full-length complement thereof; or (iv) a subsequence thereof; under very low to very high stringency conditions. Molecules to which the nucleic acid probe hybridizes under these conditions can be detected using, for example, X-ray film or any other detection means known in the art.

In one aspect, the nucleic acid probe is nucleotides 1 to 100, nucleotides 1 to 200, nucleotides 1 to 300, or nucleotides 1 to 400 of SEQ ID NO: 1 or SEQ ID NO: 3. In another aspect, the nucleic acid probe is a polynucleotide that encodes the polypeptide of SEQ ID NO: 2; the mature polypeptide thereof; or a fragment thereof. In another aspect, the nucleic acid probe is SEQ ID NO: 1 or SEQ ID NO: 3. In another aspect, the nucleic acid probe is the polynucleotide in SEQ ID NO: 1 or SEQ ID NO: 3, nucleotides 1 to 1800.

In another embodiment, the present invention relates to an isolated polypeptide having glucose dehydrogenase activity encoded by a polynucleotide having a sequence identity to the mature polypeptide coding sequence of SEQ ID NO: 1 or SEQ ID NO: 3 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%.

In another embodiment, the present invention relates to variants of the mature polypeptide of SEQ ID NO: 2 comprising a substitution, deletion, and/or insertion at one or more (e.g., several) positions. In an embodiment, the number of amino acid substitutions, deletions and/or insertions introduced into the mature polypeptide of SEQ ID NO: 2 is not more than 10, e.g., 1, 2, 3, 4, 5, 6, 7, 8 or 9. The amino acid changes may be of a minor nature, that is conservative amino acid substitutions or insertions that do not significantly affect the folding and/or activity of the protein; small deletions, typically of 1-30 amino acids; small amino- or carboxyl-terminal extensions, such as an amino-terminal methionine residue; a small linker peptide of up to 20-25 residues; or a small extension that facilitates purification by changing net charge or another function, such as a poly-histidine tract, an antigenic epitope or a binding domain.

Examples of conservative substitutions are within the groups of basic amino acids (arginine, lysine and histidine), acidic amino acids (glutamic acid and aspartic acid), polar amino acids (glutamine and asparagine), hydrophobic amino acids (leucine, isoleucine and valine), aromatic amino acids (phenylalanine, tryptophan and tyrosine), and small amino acids (glycine, alanine, serine, threonine and methionine). Amino acid substitutions that do not generally alter specific activity are known in the art and are described, for example, by H. Neurath and R. L. Hill, 1979, In, The Proteins, Academic Press, New York. Common substitutions are Ala/Ser, Val/Ile, Asp/Glu, Thr/Ser, Ala/Gly, Ala/Thr, Ser/Asn, Ala/Val, Ser/Gly, Tyr/Phe, Ala/Pro, Lys/Arg, Asp/Asn, Leu/Ile, Leu/Val, Ala/Glu, and Asp/Gly.

Alternatively, the amino acid changes are of such a nature that the physico-chemical properties of the polypeptides are altered. For example, amino acid changes may improve the thermal stability of the polypeptide, alter the substrate specificity, change the pH optimum, and the like.

Essential amino acids in a polypeptide can be identified according to procedures known in the art, such as site-directed mutagenesis or alanine-scanning mutagenesis (Cunningham and Wells, 1989, Science 244: 1081-1085). In the latter technique, single alanine mutations are introduced at every residue in the molecule, and the resultant mutant molecules are tested for glucose dehydrogenase activity to identify amino acid residues that are critical to the activity of the molecule. See also, Hilton et al., 1996, J. Biol. Chem. 271: 4699-4708. The active site of the enzyme or other biological interaction can also be determined by physical analysis of structure, as determined by such techniques as nuclear magnetic resonance, crystallography, electron diffraction, or photoaffinity labeling, in conjunction with mutation of putative contact site amino acids. See, for example, de Vos et al., 1992, Science 255: 306-312; Smith et al., 1992, J. Mol. Biol. 224: 899-904; Wlodaver et al., 1992, FEBS Lett. 309: 59-64. The identity of essential amino acids can also be inferred from an alignment with a related polypeptide.

Single or multiple amino acid substitutions, deletions, and/or insertions can be made and tested using known methods of mutagenesis, recombination, and/or shuffling, followed by a relevant screening procedure, such as those disclosed by Reidhaar-Olson and Sauer, 1988, Science 241: 53-57; Bowie and Sauer, 1989, Proc. Natl. Acad. Sci. USA 86: 2152-2156; WO95/17413; or WO95/22625. Other methods that can be used include error-prone PCR, phage display (e.g., Lowman et al., 1991, Biochemistry 30: 10832-10837; U.S. Pat. No. 5,223,409; WO92/06204), and region-directed mutagenesis (Derbyshire et al., 1986, Gene 46: 145; Ner et al., 1988, DNA 7: 127).

Mutagenesis/shuffling methods can be combined with high-throughput, automated screening methods to detect activity of cloned, mutagenized polypeptides expressed by host cells (Ness et al., 1999, Nature Biotechnology 17: 893-896). Mutagenized DNA molecules that encode active polypeptides can be recovered from the host cells and rapidly sequenced using standard methods in the art. These methods allow the rapid determination of the importance of individual amino acid residues in a polypeptide.

The polypeptide may be a hybrid polypeptide in which a region of one polypeptide is fused at the N-terminus or the C-terminus of a region of another polypeptide.

The polypeptide may be a fusion polypeptide or cleavable fusion polypeptide in which another polypeptide is fused at the N-terminus or the C-terminus of the polypeptide of the present invention. A fusion polypeptide is produced by fusing a polynucleotide encoding another polypeptide to a polynucleotide of the present invention. Techniques for producing fusion polypeptides are known in the art, and include ligating the coding sequences encoding the polypeptides so that they are in frame and that expression of the fusion polypeptide is under control of the same promoter(s) and terminator. Fusion polypeptides may also be constructed using intein technology in which fusion polypeptides are created post-translationally (Cooper et al., 1993, EMBO J. 12: 2575-2583; Dawson et al., 1994, Science 266: 776-779).

A fusion polypeptide can further comprise a cleavage site between the two polypeptides. Upon secretion of the fusion protein, the site is cleaved releasing the two polypeptides. Examples of cleavage sites include, but are not limited to, the sites disclosed in Martin et al., 2003, J. Ind. Microbiol. Biotechnol. 3: 568-576; Svetina et al., 2000, J. Biotechnol. 76: 245-251; Rasmussen-Wilson et al., 1997, Appl. Environ. Microbiol. 63: 3488-3493; Ward et al., 1995, Biotechnology 13: 498-503; and Contreras et al., 1991, Biotechnology 9: 378-381; Eaton et al., 1986, Biochemistry 25: 505-512; Collins-Racie et al., 1995, Biotechnology 13: 982-987; Carter et al., 1989, Proteins: Structure, Function, and Genetics 6: 240-248; and Stevens, 2003, Drug Discovery World 4: 35-48.

### Sources of Polypeptides Having Glucose Dehydrogenase Activity

A polypeptide having glucose dehydrogenase activity of the present invention may be obtained from microorganisms of any genus. For purposes of the present invention, the term “obtained from” as used herein in connection with a given source shall mean that the polypeptide encoded by a polynucleotide is produced by the source or by a strain in which the polynucleotide from the source has been inserted. In one aspect, the polypeptide obtained from a given source is secreted extracellularly.

The polypeptide may be a fungal polypeptide. For example, the polypeptide may be a yeast polypeptide such as a Candida, Kluyveromyces, Pichia, Saccharomyces, Schizosaccharomyces, or Yarrowia polypeptide; or a filamentous fungal polypeptide such as an Acremonium, Agaricus, Alternaria, Aspergillus, Aureobasidium, Botryospaeria, Ceriporiopsis, Chaetomidium, Chrysosporium, Claviceps, Cochliobolus, Coprinopsis, Coptotermes, Corynascus, Cryphonectria, Cryptococcus, Diplodia, Exidia, Filibasidium, Fusarium, Gibberella, Holomastigotoides, Humicola, Irpex, Lentinula, Leptospaeria, Magnaporthe, Melanocarpus, Meripilus, Mucor, Myceliophthora, Neocallimastix, Neurospora, Paecilomyces, Penicillium, Phanerochaete, Piromyces, Poitrasia, Pseudoplectania, Pseudotrichonympha, Rhizomucor, Schizophyllum, Scytalidium, Talaromyces, Thermoascus, Thielavia, Tolypocladium, Trichoderma, Trichophaea, Verticillium, Volvariella, or Xylaria polypeptide.

In another aspect, the polypeptide is a Saccharomyces carlsbergensis, Saccharomyces cerevisiae, Saccharomyces diastaticus, Saccharomyces douglasfi, Saccharomyces kluyveri, Saccharomyces norbensis, or Saccharomyces oviformis polypeptide.

In another aspect, the polypeptide is an Acremonium cellulolyticus, Aspergillus aculeatus, Aspergillus awamori, Aspergillus foetidus, Aspergillus fumigatus, Aspergillus japonicus, Aspergillus nidulans, Aspergillus niger, Aspergillus oryzae, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola grisea, Humicola insolens, Humicola lanuginosa, Irpex lacteus, Mucor miehei, Myceliophthora thermophila, Neurospora crassa, Penicillium funiculosum, Penicillium purpurogenum, Phanerochaete chrysosporium, Thielavia achromatica, Thielavia albomyces, Thielavia albopilosa, Thielavia australeinsis, Thielavia fimeti, Thielavia microspora, Thielavia ovispora, Thielavia peruviana, Thielavia setosa, Thielavia spededonium, Thielavia subthermophila, Thielavia terrestris, Trichoderma harzianum, Trichoderma koningii, Trichoderma longibrachiatum, Trichoderma reesei, or Trichoderma viride polypeptide.

In another aspect, the polypeptide is a Glomerella sp. polypeptide, e.g., a Glomerella cingulata polypeptide, e.g., a polypeptide obtained from Glomerella cingulata DSM 62728.

It will be understood that for the aforementioned species, the invention encompasses both the perfect and imperfect states, and other taxonomic equivalents, e.g., anamorphs, regardless of the species name by which they are known. Those skilled in the art will readily recognize the identity of appropriate equivalents.

Strains of these species are readily accessible to the public in a number of culture collections, such as the American Type Culture Collection (ATCC), Deutsche Sammlung von Mikroorganismen and Zellkulturen GmbH (DSMZ), Centraalbureau Voor Schimmelcultures (CBS), and Agricultural Research Service Patent Culture Collection, Northern Regional Research Center (NRRL).

The polypeptide may be identified and obtained from other sources including microorganisms isolated from nature (e.g., soil, composts, water, etc.) or DNA samples obtained directly from natural materials (e.g., soil, composts, water, etc.) using the above-mentioned probes. Techniques for isolating microorganisms and DNA directly from natural habitats are well known in the art. A polynucleotide encoding the polypeptide may then be obtained by similarly screening a genomic DNA or cDNA library of another microorganism or mixed DNA sample.

Once a polynucleotide encoding a polypeptide has been detected with the probe(s), the polynucleotide can be isolated or cloned by utilizing techniques that are known to those of ordinary skill in the art (see, e.g., Sambrook et al., 1989, supra).

### Polynucleotides

The present invention also relates to isolated polynucleotides encoding a polypeptide of the present invention, as described herein.

The techniques used to isolate or clone a polynucleotide are known in the art and include isolation from genomic DNA or cDNA, or a combination thereof. The cloning of the polynucleotides from genomic DNA can be effected, e.g., by using the well known polymerase chain reaction (PCR) or antibody screening of expression libraries to detect cloned DNA fragments with shared structural features. See, e.g., Innis et al., 1990, PCR: A Guide to Methods and Application, Academic Press, New York. Other nucleic acid amplification procedures such as ligase chain reaction (LCR), ligation activated transcription (LAT) and polynucleotide-based amplification (NASBA) may be used. The polynucleotides may be cloned from a strain of a Glomerella sp., preferably from a strain of Glomerella cingulata, or a related organism and thus, for example, may be an allelic or species variant of the polypeptide encoding region of the polynucleotide.

Modification of a polynucleotide encoding a polypeptide of the present invention may be necessary for synthesizing polypeptides substantially similar to the polypeptide. The term “substantially similar” to the polypeptide refers to non-naturally occurring forms of the polypeptide. These polypeptides may differ in some engineered way from the polypeptide isolated from its native source, e.g., variants that differ in specific activity, thermostability, pH optimum, or the like. The variants may be constructed on the basis of the polynucleotide presented as the mature polypeptide coding sequence of SEQ ID NO: 1 or SEQ ID NO: 3, e.g., a subsequence thereof, and/or by introduction of nucleotide substitutions that do not result in a change in the amino acid sequence of the polypeptide, but which correspond to the codon usage of the host organism intended for production of the enzyme, or by introduction of nucleotide substitutions that may give rise to a different amino acid sequence. For example, the polynucleotide shown in SEQ ID NO:3 is a modification of the native polynucleotide shown in SEQ ID NO:1 wherein the codon usage of Aspergillus oryzae has been applied. Both sequences SEQ ID NO:1 and SEQ ID NO:3 encode the wild type glucose dehydrogenase of Glomerella cingulata. For a general description of nucleotide substitution, see, e.g., Ford et al., 1991, Protein Expression and Purification 2: 95-107.

### Nucleic Acid Constructs

The present invention also relates to nucleic acid constructs comprising a polynucleotide of the present invention operably linked to one or more control sequences that direct the expression of the coding sequence in a suitable host cell under conditions compatible with the control sequences.

A polynucleotide may be manipulated in a variety of ways to provide for expression of the polypeptide. Manipulation of the polynucleotide prior to its insertion into a vector may be desirable or necessary depending on the expression vector. The techniques for modifying polynucleotides utilizing recombinant DNA methods are well known in the art.

The control sequence may be a promoter, a polynucleotide that is recognized by a host cell for expression of a polynucleotide encoding a polypeptide of the present invention. The promoter contains transcriptional control sequences that mediate the expression of the polypeptide. The promoter may be any polynucleotide that shows transcriptional activity in the host cell including mutant, truncated, and hybrid promoters, and may be obtained from genes encoding extracellular or intracellular polypeptides either homologous or heterologous to the host cell.

Examples of suitable promoters for directing transcription of the nucleic acid constructs of the present invention in a bacterial host cell are the promoters obtained from the Bacillus amyloliquefaciens alpha-amylase gene (amyQ), Bacillus licheniformis alpha-amylase gene (amyL), Bacillus licheniformis penicillinase gene (penP), Bacillus stearothermophilus maltogenic amylase gene (amyM), Bacillus subtilis levansucrase gene (sacB), Bacillus subtilis xylA and xylB genes, Bacillus thuringiensis cryIIIA gene (Agaisse and Lereclus, 1994, Molecular Microbiology 13: 97-107), E. coli lac operon, E. coli trc promoter (Egon et al., 1988, Gene 69: 301-315), Streptomyces coelicolor agarase gene (dagA), and prokaryotic beta-lactamase gene (Villa-Kamaroff et al., 1978, Proc. Natl. Acad. Sci. USA 75: 3727-3731), as well as the tac promoter (DeBoer et al., 1983, Proc. Natl. Acad. Sci. USA 80: 21-25). Further promoters are described in “Useful proteins from recombinant bacteria” in Gilbert et al., 1980, Scientific American 242: 74-94; and in Sambrook et al., 1989, supra. Examples of tandem promoters are disclosed in WO99/43835.

Examples of suitable promoters for directing transcription of the nucleic acid constructs of the present invention in a filamentous fungal host cell are promoters obtained from the genes for Aspergillus nidulans acetamidase, Aspergillus niger neutral alpha-amylase, Aspergillus niger acid stable alpha-amylase, Aspergillus niger or Aspergillus awamori glucoamylase (glaA), Aspergillus oryzae TAKA amylase, Aspergillus oryzae alkaline protease, Aspergillus oryzae triose phosphate isomerase, Fusarium oxysporum trypsin-like protease (WO96/00787), Fusarium venenatum amyloglucosidase (WO2000/56900), Fusarium venenatum Daria (WO2000/56900), Fusarium venenatum Quinn (WO2000/56900), Rhizomucor miehei lipase, Rhizomucor miehei aspartic proteinase, Trichoderma reesei beta-glucosidase, Trichoderma reesei cellobiohydrolase I, Trichoderma reesei cellobiohydrolase II, Trichoderma reesei endoglucanase I, Trichoderma reesei endoglucanase II, Trichoderma reesei endoglucanase III, Trichoderma reesei endoglucanase IV, Trichoderma reesei endoglucanase V, Trichoderma reesei xylanase I, Trichoderma reesei xylanase II, Trichoderma reesei beta-xylosidase, as well as the NA2-tpi promoter (a modified promoter from an Aspergillus neutral alpha-amylase gene in which the untranslated leader has been replaced by an untranslated leader from an Aspergillus triose phosphate isomerase gene; non-limiting examples include modified promoters from an Aspergillus niger neutral alpha-amylase gene in which the untranslated leader has been replaced by an untranslated leader from an Aspergillus nidulans or Aspergillus oryzae triose phosphate isomerase gene); and mutant, truncated, and hybrid promoters thereof.

In a yeast host, useful promoters are obtained from the genes for Saccharomyces cerevisiae enolase (ENO-1), Saccharomyces cerevisiae galactokinase (GAL1), Saccharomyces cerevisiae alcohol dehydrogenase/glyceraldehyde-3-phosphate dehydrogenase (ADH1, ADH2/GAP), Saccharomyces cerevisiae triose phosphate isomerase (TPI), Saccharomyces cerevisiae metallothionein (CUP1), and Saccharomyces cerevisiae 3-phosphoglycerate kinase. Other useful promoters for yeast host cells are described by Romanos et al., 1992, Yeast 8: 423-488.

The control sequence may also be a transcription terminator, which is recognized by a host cell to terminate transcription. The terminator is operably linked to the 3′-terminus of the polynucleotide encoding the polypeptide. Any terminator that is functional in the host cell may be used in the present invention.

Preferred terminators for bacterial host cells are obtained from the genes for Bacillus clausii alkaline protease (aprH), Bacillus licheniformis alpha-amylase (amyL), and Escherichia coli ribosomal RNA (rrnB).

Preferred terminators for filamentous fungal host cells are obtained from the genes for Aspergillus nidulans anthranilate synthase, Aspergillus niger glucoamylase, Aspergillus niger alpha-glucosidase, Aspergillus oryzae TAKA amylase, and Fusarium oxysporum trypsin-like protease.

Preferred terminators for yeast host cells are obtained from the genes for Saccharomyces cerevisiae enolase, Saccharomyces cerevisiae cytochrome C (CYC1), and Saccharomyces cerevisiae glyceraldehyde-3-phosphate dehydrogenase. Other useful terminators for yeast host cells are described by Romanos et al., 1992, supra.

The control sequence may also be an mRNA stabilizer region downstream of a promoter and upstream of the coding sequence of a gene which increases expression of the gene.

Examples of suitable mRNA stabilizer regions are obtained from a Bacillus thuringiensis cryIIIA gene (WO94/25612) and a Bacillus subtilis SP82 gene (Hue et al., 1995, Journal of Bacteriology 177: 3465-3471).

The control sequence may also be a leader, a nontranslated region of an mRNA that is important for translation by the host cell. The leader is operably linked to the 5′-terminus of the polynucleotide encoding the polypeptide. Any leader that is functional in the host cell may be used.

Preferred leaders for filamentous fungal host cells are obtained from the genes for Aspergillus oryzae TAKA amylase and Aspergillus nidulans triose phosphate isomerase.

Suitable leaders for yeast host cells are obtained from the genes for Saccharomyces cerevisiae enolase (ENO-1), Saccharomyces cerevisiae 3-phosphoglycerate kinase, Saccharomyces cerevisiae alpha-factor, and Saccharomyces cerevisiae alcohol dehydrogenase/glyceraldehyde-3-phosphate dehydrogenase (ADH2/GAP).

The control sequence may also be a polyadenylation sequence, a sequence operably linked to the 3′-terminus of the polynucleotide and, when transcribed, is recognized by the host cell as a signal to add polyadenosine residues to transcribed mRNA. Any polyadenylation sequence that is functional in the host cell may be used.

Preferred polyadenylation sequences for filamentous fungal host cells are obtained from the genes for Aspergillus nidulans anthranilate synthase, Aspergillus niger glucoamylase, Aspergillus niger alpha-glucosidase Aspergillus oryzae TAKA amylase, and Fusarium oxysporum trypsin-like protease.

Useful polyadenylation sequences for yeast host cells are described by Guo and Sherman, 1995, Mol. Cellular Biol. 15: 5983-5990.

The control sequence may also be a signal peptide coding region that encodes a signal peptide linked to the N-terminus of a polypeptide and directs the polypeptide into the cell's secretory pathway. The 5′-end of the coding sequence of the polynucleotide may inherently contain a signal peptide coding sequence naturally linked in translation reading frame with the segment of the coding sequence that encodes the polypeptide. Alternatively, the 5′-end of the coding sequence may contain a signal peptide coding sequence that is foreign to the coding sequence. A foreign signal peptide coding sequence may be required where the coding sequence does not naturally contain a signal peptide coding sequence. Alternatively, a foreign signal peptide coding sequence may simply replace the natural signal peptide coding sequence in order to enhance secretion of the polypeptide. However, any signal peptide coding sequence that directs the expressed polypeptide into the secretory pathway of a host cell may be used.

Effective signal peptide coding sequences for bacterial host cells are the signal peptide coding sequences obtained from the genes for Bacillus NCIB 11837 maltogenic amylase, Bacillus licheniformis subtilisin, Bacillus licheniformis beta-lactamase, Bacillus stearothermophilus alpha-amylase, Bacillus stearothermophilus neutral proteases (nprT, nprS, nprM), and Bacillus subtilis prsA. Further signal peptides are described by Simonen and Palva, 1993, Microbiological Reviews 57: 109-137.

Effective signal peptide coding sequences for filamentous fungal host cells are the signal peptide coding sequences obtained from the genes for Aspergillus niger neutral amylase, Aspergillus niger glucoamylase, Aspergillus oryzae TAKA amylase, Humicola insolens cellulase, Humicola insolens endoglucanase V, Humicola lanuginosa lipase, and Rhizomucor miehei aspartic proteinase.

Useful signal peptides for yeast host cells are obtained from the genes for Saccharomyces cerevisiae alpha-factor and Saccharomyces cerevisiae invertase. Other useful signal peptide coding sequences are described by Romanos et al., 1992, supra.

The control sequence may also be a propeptide coding sequence that encodes a propeptide positioned at the N-terminus of a polypeptide. The resultant polypeptide is known as a proenzyme or propolypeptide (or a zymogen in some cases). A propolypeptide is generally inactive and can be converted to an active polypeptide by catalytic or autocatalytic cleavage of the propeptide from the propolypeptide. The propeptide coding sequence may be obtained from the genes for Bacillus subtilis alkaline protease (aprE), Bacillus subtilis neutral protease (nprT), Myceliophthora thermophila laccase (WO95/33836), Rhizomucor miehei aspartic proteinase, and Saccharomyces cerevisiae alpha-factor.

Where both signal peptide and propeptide sequences are present, the propeptide sequence is positioned next to the N-terminus of a polypeptide and the signal peptide sequence is positioned next to the N-terminus of the propeptide sequence.

It may also be desirable to add regulatory sequences that regulate expression of the polypeptide relative to the growth of the host cell. Examples of regulatory systems are those that cause expression of the gene to be turned on or off in response to a chemical or physical stimulus, including the presence of a regulatory compound. Regulatory systems in prokaryotic systems include the lac, tac, and trp operator systems. In yeast, the ADH2 system or GAL1 system may be used. In filamentous fungi, the Aspergillus niger glucoamylase promoter, Aspergillus oryzae TAKA alpha-amylase promoter, and Aspergillus oryzae glucoamylase promoter may be used. Other examples of regulatory sequences are those that allow for gene amplification. In eukaryotic systems, these regulatory sequences include the dihydrofolate reductase gene that is amplified in the presence of methotrexate, and the metallothionein genes that are amplified with heavy metals. In these cases, the polynucleotide encoding the polypeptide would be operably linked with the regulatory sequence.

### Expression Vectors

The present invention also relates to recombinant expression vectors comprising a polynucleotide of the present invention, a promoter, and transcriptional and translational stop signals. The various nucleotide and control sequences may be joined together to produce a recombinant expression vector that may include one or more convenient restriction sites to allow for insertion or substitution of the polynucleotide encoding the polypeptide at such sites. Alternatively, the polynucleotide may be expressed by inserting the polynucleotide or a nucleic acid construct comprising the polynucleotide into an appropriate vector for expression. In creating the expression vector, the coding sequence is located in the vector so that the coding sequence is operably linked with the appropriate control sequences for expression.

The recombinant expression vector may be any vector (e.g., a plasmid or virus) that can be conveniently subjected to recombinant DNA procedures and can bring about expression of the polynucleotide. The choice of the vector will typically depend on the compatibility of the vector with the host cell into which the vector is to be introduced. The vector may be a linear or closed circular plasmid.

The vector may be an autonomously replicating vector, i.e., a vector that exists as an extrachromosomal entity, the replication of which is independent of chromosomal replication, e.g., a plasmid, an extrachromosomal element, a minichromosome, or an artificial chromosome. The vector may contain any means for assuring self-replication. Alternatively, the vector may be one that, when introduced into the host cell, is integrated into the genome and replicated together with the chromosome(s) into which it has been integrated. Furthermore, a single vector or plasmid or two or more vectors or plasmids that together contain the total DNA to be introduced into the genome of the host cell, or a transposon, may be used.

The vector preferably contains one or more selectable markers that permit easy selection of transformed, transfected, transduced, or the like cells. A selectable marker is a gene the product of which provides for biocide or viral resistance, resistance to heavy metals, prototrophy to auxotrophs, and the like.

Examples of bacterial selectable markers are Bacillus licheniformis or Bacillus subtilis dal genes, or markers that confer antibiotic resistance such as ampicillin, chloramphenicol, kanamycin, neomycin, spectinomycin, or tetracycline resistance. Suitable markers for yeast host cells include, but are not limited to, ADE2, HIS3, LEU2, LYS2, MET3, TRP1, and URA3. Selectable markers for use in a filamentous fungal host cell include, but are not limited to, amdS (acetamidase), argB (ornithine carbamoyltransferase), bar (phosphinothricin acetyltransferase), hph (hygromycin phosphotransferase), niaD (nitrate reductase), pyrG (orotidine-5′-phosphate decarboxylase), sC (sulfate adenyltransferase), and trpC (anthranilate synthase), as well as equivalents thereof. Preferred for use in an Aspergillus cell are Aspergillus nidulans or Aspergillus oryzae amdS and pyrG genes and a Streptomyces hygroscopicus bar gene.

The vector preferably contains an element(s) that permits integration of the vector into the host cell's genome or autonomous replication of the vector in the cell independent of the genome.

For integration into the host cell genome, the vector may rely on the polynucleotide's sequence encoding the polypeptide or any other element of the vector for integration into the genome by homologous or non-homologous recombination. Alternatively, the vector may contain additional polynucleotides for directing integration by homologous recombination into the genome of the host cell at a precise location(s) in the chromosome(s). To increase the likelihood of integration at a precise location, the integrational elements should contain a sufficient number of nucleic acids, such as 100 to 10,000 base pairs, 400 to 10,000 base pairs, and 800 to 10,000 base pairs, which have a high degree of sequence identity to the corresponding target sequence to enhance the probability of homologous recombination. The integrational elements may be any sequence that is homologous with the target sequence in the genome of the host cell. Furthermore, the integrational elements may be non-encoding or encoding polynucleotides. On the other hand, the vector may be integrated into the genome of the host cell by non-homologous recombination.

For autonomous replication, the vector may further comprise an origin of replication enabling the vector to replicate autonomously in the host cell in question. The origin of replication may be any plasmid replicator mediating autonomous replication that functions in a cell. The term “origin of replication” or “plasmid replicator” means a polynucleotide that enables a plasmid or vector to replicate in vivo.

Examples of bacterial origins of replication are the origins of replication of plasmids pBR322, pUC19, pACYC177, and pACYC184 permitting replication in E. coli, and pUB110, pE194, pTA1060, and pAMβ1 permitting replication in Bacillus.

Examples of origins of replication for use in a yeast host cell are the 2 micron origin of replication, ARS1, ARS4, the combination of ARS1 and CEN3, and the combination of ARS4 and CEN6.

Examples of origins of replication useful in a filamentous fungal cell are AMA1 and ANS1 (Gems et al., 1991, Gene 98: 61-67; Cullen et al., 1987, Nucleic Acids Res. 15: 9163-9175; WO2000/24883). Isolation of the AMA1 gene and construction of plasmids or vectors comprising the gene can be accomplished according to the methods disclosed in WO2000/24883.

More than one copy of a polynucleotide of the present invention may be inserted into a host cell to increase production of a polypeptide. An increase in the copy number of the polynucleotide can be obtained by integrating at least one additional copy of the sequence into the host cell genome or by including an amplifiable selectable marker gene with the polynucleotide where cells containing amplified copies of the selectable marker gene, and thereby additional copies of the polynucleotide, can be selected for by cultivating the cells in the presence of the appropriate selectable agent.

The procedures used to ligate the elements described above to construct the recombinant expression vectors of the present invention are well known to one skilled in the art (see, e.g., Sambrook et al., 1989, supra).

### Host Cells

The present invention also relates to recombinant host cells, comprising a polynucleotide of the present invention operably linked to one or more control sequences that direct the production of a polypeptide of the present invention. A construct or vector comprising a polynucleotide is introduced into a host cell so that the construct or vector is maintained as a chromosomal integrant or as a self-replicating extra-chromosomal vector as described earlier. The term “host cell” encompasses any progeny of a parent cell that is not identical to the parent cell due to mutations that occur during replication. The choice of a host cell will to a large extent depend upon the gene encoding the polypeptide and its source.

The host cell may be any cell useful in the recombinant production of a polypeptide of the present invention, e.g., a prokaryote or a eukaryote.

The prokaryotic host cell may be any Gram-positive or Gram-negative bacterium. Gram-positive bacteria include, but are not limited to, Bacillus, Clostridium, Enterococcus, Geobacillus, Lactobacillus, Lactococcus, Oceanobacillus, Staphylococcus, Streptococcus, and Streptomyces. Gram-negative bacteria include, but are not limited to, Campylobacter, E. coli, Flavobacterium, Fusobacterium, Helicobacter, Ilyobacter, Neisseria, Pseudomonas, Salmonella, and Ureaplasma.

The host cell may also be a eukaryote, such as a mammalian, insect, plant, or fungal cell.

The host cell may be a fungal cell. “Fungi” as used herein includes the phyla Ascomycota, Basidiomycota, Chytridiomycota, and Zygomycota as well as the Oomycota and all mitosporic fungi (as defined by Hawksworth et al., In, Ainsworth and Bisby's Dictionary of The Fungi, 8th edition, 1995, CAB International, University Press, Cambridge, UK). The fungal host cell may be a yeast cell. “Yeast” as used herein includes ascosporogenous yeast (Endomycetales), basidiosporogenous yeast, and yeast belonging to the Fungi Imperfecti (Blastomycetes). Since the classification of yeast may change in the future, for the purposes of this invention, yeast shall be defined as described in Biology and Activities of Yeast (Skinner, Passmore, and Davenport, editors, Soc. App. Bacteriol. Symposium Series No. 9, 1980).

The yeast host cell may be a Candida, Hansenula, Kluyveromyces, Pichia, Saccharomyces, Schizosaccharomyces, or Yarrowia cell, such as a Kluyveromyces lactis, Saccharomyces carlsbergensis, Saccharomyces cerevisiae, Saccharomyces diastaticus, Saccharomyces douglasii, Saccharomyces kluyveri, Saccharomyces norbensis, Saccharomyces oviformis, or Yarrowia lipolytica cell.

The fungal host cell may be a filamentous fungal cell. “Filamentous fungi” include all filamentous forms of the subdivision Eumycota and Oomycota (as defined by Hawksworth et al., 1995, supra). The filamentous fungi are generally characterized by a mycelial wall composed of chitin, cellulose, glucan, chitosan, mannan, and other complex polysaccharides. Vegetative growth is by hyphal elongation and carbon catabolism is obligately aerobic. In contrast, vegetative growth by yeasts such as Saccharomyces cerevisiae is by budding of a unicellular thallus and carbon catabolism may be fermentative.

The filamentous fungal host cell may be an Acremonium, Aspergillus, Aureobasidium,

Bjerkandera, Ceriporiopsis, Chrysosporium, Coprinus, Coriolus, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes, or Trichoderma cell.

For example, the filamentous fungal host cell may be an Aspergillus awamori, Aspergillus foetidus, Aspergillus fumigatus, Aspergillus japonicus, Aspergillus nidulans, Aspergillus niger, Aspergillus oryzae, Bjerkandera adusta, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Coprinus cinereus, Coriolus hirsutus, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola insolens, Humicola lanuginosa, Mucor miehei, Myceliophthora thermophila, Neurospora crassa, Penicillium purpurogenum, Phanerochaete chrysosporium, Phlebia radiata, Pleurotus eryngii, Thielavia terrestris, Trametes villosa, Trametes versicolor, Trichoderma harzianum, Trichoderma koningii, Trichoderma longibrachiatum, Trichoderma reesei, or Trichoderma viride cell.

Fungal cells may be transformed by a process involving protoplast formation, transformation of the protoplasts, and regeneration of the cell wall in a manner known per se. Suitable procedures for transformation of Aspergillus and Trichoderma host cells are described in EP 238023, Yelton et al., 1984, Proc. Natl. Acad. Sci. USA 81: 1470-1474, and Christensen et al., 1988, Bio/Technology 6: 1419-1422. Suitable methods for transforming Fusarium species are described by Malardier et al., 1989, Gene 78: 147-156, and WO96/00787. Yeast may be transformed using the procedures described by Becker and Guarente, In Abelson, J. N. and Simon, M. I., editors, Guide to Yeast Genetics and Molecular Biology, Methods in Enzymology, Volume 194, pp 182-187, Academic Press, Inc., New York; Ito et al., 1983, J. Bacteriol. 153: 163; and Hinnen et al., 1978, Proc. Natl. Acad. Sci. USA 75: 1920.

### Methods of Production

The present invention also relates to methods of producing a polypeptide of the present invention, comprising (a) cultivating a cell, which in its wild-type form produces the polypeptide, under conditions conducive for production of the polypeptide; and (b) recovering the polypeptide. In a preferred aspect, the cell is an Aspergillus cell or a Trichoderma cell. In a more preferred aspect, the cell is an Aspergillus niger or an A. oryzae cell.

The present invention also relates to methods of producing a polypeptide of the present invention, comprising (a) cultivating a recombinant host cell of the present invention under conditions conducive for production of the polypeptide; and (b) recovering the polypeptide.

The host cells are cultivated in a nutrient medium suitable for production of the polypeptide using methods known in the art. For example, the cell may be cultivated by shake flask cultivation, or small-scale or large-scale fermentation (including continuous, batch, fed-batch, or solid state fermentations) in laboratory or industrial fermentors performed in a suitable medium and under conditions allowing the polypeptide to be expressed and/or isolated. The cultivation takes place in a suitable nutrient medium comprising carbon and nitrogen sources and inorganic salts, using procedures known in the art. Suitable media are available from commercial suppliers or may be prepared according to published compositions (e.g., in catalogues of the American Type Culture Collection). If the polypeptide is secreted into the nutrient medium, the polypeptide can be recovered directly from the medium. If the polypeptide is not secreted, it can be recovered from cell lysates.

The polypeptide may be detected using methods known in the art that are specific for the polypeptides. These detection methods include, but are not limited to, use of specific antibodies, formation of an enzyme product, or disappearance of an enzyme substrate. For example, an enzyme assay may be used to determine the activity of the polypeptide.

The polypeptide may be recovered using methods known in the art. For example, the polypeptide may be recovered from the nutrient medium by conventional procedures including, but not limited to, collection, centrifugation, filtration, extraction, spray-drying, evaporation, or precipitation.

The polypeptide may be purified by a variety of procedures known in the art including, but not limited to, chromatography (e.g., ion exchange, affinity, hydrophobic, chromatofocusing, and size exclusion), electrophoretic procedures (e.g., preparative isoelectric focusing), differential solubility (e.g., ammonium sulfate precipitation), SDS-PAGE, or extraction (see, e.g., Protein Purification, Janson and Ryden, editors, VCH Publishers, New York, 1989) to obtain substantially pure polypeptides.

In an alternative aspect, the polypeptide is not recovered, but rather a host cell of the present invention expressing the polypeptide is used as a source of the polypeptide.

### Plants

The present invention also relates to isolated plants, e.g., a transgenic plant, plant part, or plant cell, comprising a polynucleotide of the present invention so as to express and produce a polypeptide or domain in recoverable quantities. The polypeptide or domain may be recovered from the plant or plant part. Alternatively, the plant or plant part containing the polypeptide or domain may be used as such for improving the quality of a food or feed, e.g., improving nutritional value, palatability, and rheological properties, or to destroy an antinutritive factor.

The transgenic plant can be dicotyledonous (a dicot) or monocotyledonous (a monocot). Examples of monocot plants are grasses, such as meadow grass (blue grass, Poa), forage grass such as Festuca, Lolium, temperate grass, such as Agrostis, and cereals, e.g., wheat, oats, rye, barley, rice, sorghum, and maize (corn).

Examples of dicot plants are tobacco, legumes, such as lupins, potato, sugar beet, pea, bean and soybean, and cruciferous plants (family Brassicaceae), such as cauliflower, rape seed, and the closely related model organism Arabidopsis thaliana.

Examples of plant parts are stem, callus, leaves, root, fruits, seeds, and tubers as well as the individual tissues comprising these parts, e.g., epidermis, mesophyll, parenchyme, vascular tissues, meristems. Specific plant cell compartments, such as chloroplasts, apoplasts, mitochondria, vacuoles, peroxisomes and cytoplasm are also considered to be a plant part. Furthermore, any plant cell, whatever the tissue origin, is considered to be a plant part. Likewise, plant parts such as specific tissues and cells isolated to facilitate the utilization of the invention are also considered plant parts, e.g., embryos, endosperms, aleurone and seed coats.

Also included within the scope of the present invention are the progeny of such plants, plant parts, and plant cells.

The transgenic plant or plant cell expressing the polypeptide or domain may be constructed in accordance with methods known in the art. In short, the plant or plant cell is constructed by incorporating one or more expression constructs encoding the polypeptide or domain into the plant host genome or chloroplast genome and propagating the resulting modified plant or plant cell into a transgenic plant or plant cell.

The expression construct is conveniently a nucleic acid construct that comprises a polynucleotide encoding a polypeptide or domain operably linked with appropriate regulatory sequences required for expression of the polynucleotide in the plant or plant part of choice. Furthermore, the expression construct may comprise a selectable marker useful for identifying plant cells into which the expression construct has been integrated and DNA sequences necessary for introduction of the construct into the plant in question (the latter depends on the DNA introduction method to be used).

The choice of regulatory sequences, such as promoter and terminator sequences and optionally signal or transit sequences, is determined, for example, on the basis of when, where, and how the polypeptide or domain is desired to be expressed. For instance, the expression of the gene encoding a polypeptide or domain may be constitutive or inducible, or may be developmental, stage or tissue specific, and the gene product may be targeted to a specific tissue or plant part such as seeds or leaves. Regulatory sequences are, for example, described by Tague et al., 1988, Plant Physiology 86: 506.

For constitutive expression, the 35S-CaMV, the maize ubiquitin 1, or the rice actin 1 promoter may be used (Franck et al., 1980, Cell 21: 285-294; Christensen et al., 1992, Plant Mol. Biol. 18: 675-689; Zhang et al., 1991, Plant Cell 3: 1155-1165). Organ-specific promoters may be, for example, a promoter from storage sink tissues such as seeds, potato tubers, and fruits (Edwards and Coruzzi, 1990, Ann. Rev. Genet. 24: 275-303), or from metabolic sink tissues such as meristems (Ito et al., 1994, Plant Mol. Biol. 24: 863-878), a seed specific promoter such as the glutelin, prolamin, globulin, or albumin promoter from rice (Wu et al., 1998, Plant Cell Physiol. 39: 885-889), a Vicia faba promoter from the legumin B4 and the unknown seed protein gene from Vicia faba (Conrad et al., 1998, J. Plant Physiol. 152: 708-711), a promoter from a seed oil body protein (Chen et al., 1998, Plant Cell Physiol. 39: 935-941), the storage protein napA promoter from Brassica napus, or any other seed specific promoter known in the art, e.g., as described in WO91/14772. Furthermore, the promoter may be a leaf specific promoter such as the rbcs promoter from rice or tomato (Kyozuka et al., 1993, Plant Physiol. 102: 991-1000), the chlorella virus adenine methyltransferase gene promoter (Mitra and Higgins, 1994, Plant Mol. Biol. 26: 85-93), the aldP gene promoter from rice (Kagaya et al., 1995, Mol. Gen. Genet. 248: 668-674), or a wound inducible promoter such as the potato pin2 promoter (Xu et al., 1993, Plant Mol. Biol. 22: 573-588). Likewise, the promoter may be induced by abiotic treatments such as temperature, drought, or alterations in salinity or induced by exogenously applied substances that activate the promoter, e.g., ethanol, oestrogens, plant hormones such as ethylene, abscisic acid, and gibberellic acid, and heavy metals.

A promoter enhancer element may also be used to achieve higher expression of a polypeptide or domain in the plant. For instance, the promoter enhancer element may be an intron that is placed between the promoter and the polynucleotide encoding a polypeptide or domain. For instance, Xu et al., 1993, supra, disclose the use of the first intron of the rice actin 1 gene to enhance expression.

The selectable marker gene and any other parts of the expression construct may be chosen from those available in the art.

The nucleic acid construct is incorporated into the plant genome according to conventional techniques known in the art, including Agrobacterium-mediated transformation, virus-mediated transformation, microinjection, particle bombardment, biolistic transformation, and electroporation (Gasser et al., 1990, Science 244: 1293; Potrykus, 1990, Bio/Technology 8: 535; Shimamoto et al., 1989, Nature 338: 274).

Agrobacterium tumefaciens-mediated gene transfer is a method for generating transgenic dicots (for a review, see Hooykas and Schilperoort, 1992, Plant Mol. Biol. 19: 15-38) and for transforming monocots, although other transformation methods may be used for these plants. A method for generating transgenic monocots is particle bombardment (microscopic gold or tungsten particles coated with the transforming DNA) of embryonic calli or developing embryos (Christou, 1992, Plant J. 2: 275-281; Shimamoto, 1994, Curr. Opin. Biotechnol. 5: 158-162; Vasil et al., 1992, Bio/Technology 10: 667-674). An alternative method for transformation of monocots is based on protoplast transformation as described by Omirulleh et al., 1993, Plant Mol. Biol. 21: 415-428. Additional transformation methods include those described in U.S. Pat. Nos. 6,395,966 and 7,151,204 (both of which are herein incorporated by reference in their entirety).

Following transformation, the transformants having incorporated the expression construct are selected and regenerated into whole plants according to methods well known in the art. Often the transformation procedure is designed for the selective elimination of selection genes either during regeneration or in the following generations by using, for example, co-transformation with two separate T-DNA constructs or site specific excision of the selection gene by a specific recombinase.

In addition to direct transformation of a particular plant genotype with a construct of the present invention, transgenic plants may be made by crossing a plant having the construct to a second plant lacking the construct. For example, a construct encoding a polypeptide or domain can be introduced into a particular plant variety by crossing, without the need for ever directly transforming a plant of that given variety. Therefore, the present invention encompasses not only a plant directly regenerated from cells which have been transformed in accordance with the present invention, but also the progeny of such plants. As used herein, progeny may refer to the offspring of any generation of a parent plant prepared in accordance with the present invention. Such progeny may include a DNA construct prepared in accordance with the present invention. Crossing results in the introduction of a transgene into a plant line by cross pollinating a starting line with a donor plant line. Non-limiting examples of such steps are described in U.S. Pat. No. 7,151,204.

Plants may be generated through a process of backcross conversion. For example, plants include plants referred to as a backcross converted genotype, line, inbred, or hybrid.

Genetic markers may be used to assist in the introgression of one or more transgenes of the invention from one genetic background into another. Marker assisted selection offers advantages relative to conventional breeding in that it can be used to avoid errors caused by phenotypic variations. Further, genetic markers may provide data regarding the relative degree of elite germplasm in the individual progeny of a particular cross. For example, when a plant with a desired trait which otherwise has a non-agronomically desirable genetic background is crossed to an elite parent, genetic markers may be used to select progeny which not only possess the trait of interest, but also have a relatively large proportion of the desired germplasm. In this way, the number of generations required to introgress one or more traits into a particular genetic background is minimized.

The present invention also relates to methods of producing a polypeptide or domain of the present invention comprising (a) cultivating a transgenic plant or a plant cell comprising a polynucleotide encoding the polypeptide or domain under conditions conducive for production of the polypeptide or domain; and (b) recovering the polypeptide or domain.

### Uses

The present invention also relates to methods for preparing a dough and/or a baked product comprising incorporating into the dough an effective amount of the glucose dehydrogenase which improve one or more properties of the dough and/or the baked product obtained from the dough relative to a dough or a baked product in which the glucose dehydrogenase is not incorporated.

The use of the glucose dehydrogenase may result in an increased strength, stability, and/or reduced stickiness of the dough, resulting in improved machinability, as well as in an increased volume, improved crumb structure, and/or softness of the baked product. The effect on the dough may be particularly advantageous when a poor quality flour is used. Improved machinability is of particular importance in connection with dough that is to be processed industrially.

The improved property may be determined by comparison of a dough and/or a baked product prepared with and without addition of the glucose dehydrogenase in accordance with the present invention. Techniques which can be used to determine improvements achieved by use of the methods of present invention are described below in the Examples. Organoleptic qualities may be evaluated using procedures well established in the baking industry, and may include, for example, the use of a panel of trained taste-testers.

In a preferred embodiment, the glucose dehydrogenase of the invention improves one or more properties of the dough or the baked product obtained from the dough. In another preferred embodiment, the glucose dehydrogenase improves one or more properties of the dough and the baked product obtained from the dough.

In a preferred embodiment, the improved property is increased strength of the dough. In another preferred embodiment, the improved property is increased elasticity of the dough. In another preferred embodiment, the improved property is increased stability of the dough. In another preferred embodiment, the improved property is reduced stickiness of the dough. In another preferred embodiment, the improved property is improved extensibility of the dough. In another preferred embodiment, the improved property is improved machinability of the dough. In another preferred embodiment, the improved property is increased volume of the baked product. In another preferred embodiment, the improved property is improved crumb structure of the baked product. In another preferred embodiment, the improved property is improved crust of the baked product. In another preferred embodiment, the improved property is improved bloom of the baked product. In another preferred embodiment, the improved property is improved softness of the baked product. In another preferred embodiment, the improved property is improved flavor of the baked product. In another preferred embodiment, the improved property is improved antistaling of the baked product.

The glucose dehydrogenase of the present invention is particularly suited for use in preparation of a baked product due to the relatively low thermal stability (see Example 6). This ensures that the enzyme is deactivated in the heat treatment during the preparation of the baked product.

The glucose dehydrogenase of the present invention may also be used in a brewing process, in a bio fuel cell, e.g., such as disclosed in US2011076736A and/or US2010319424A, in a biomass degradation process, e.g., for production of ethanol or another fermentation product, or in a blood glucose sensor, e.g., for diabetic patients as described in US2007/0105174.

### Compositions

The glucose dehydrogenase of the present invention may be provided as part of a composition. The composition may be a dough and/or bread improving additive, such as a dough conditioner, in the form of a granulate or agglomerated powder. The dough and/or bread improving additive preferably may particularly have a narrow particle size distribution with more than 95% (by weight) of the particles in the range from 25 to 500 μm.

Granulates and agglomerated powders may be prepared by conventional methods, e.g. by spraying the amylase onto a carrier in a fluid-bed granulator. The carrier may consist of particulate cores having a suitable particle size. The carrier may be soluble or insoluble, e.g. a salt (such as NaCl or sodium sulfate), a sugar (such as sucrose or lactose), a sugar alcohol (such as sorbitol), starch, rice, corn grits, or soy.

The amount of glucose dehydrogenase in the composition may be between 0.5-1000 mg polypeptide per kg dry matter, 0.5-100 mg polypeptide per kg dry matter, 0.5-50 mg polypeptide per kg dry matter, 1-25 mg polypeptide per kg dry matter, 1-15 mg polypeptide per kg dry matter, 2-10 mg/kg. Preferably the composition comprises glucose dehydrogenase in an amount of at least 0.5 mg polypeptide per kg dry matter, at least 1 mg polypeptide per kg dry matter, at least 10 mg polypeptide per kg dry matter, at least 50 mg polypeptide per kg dry matter, at least 100 mg polypeptide per kg dry matter, at least 500 mg polypeptide per kg dry matter, or even at least 1000 mg polypeptide per kg dry matter.

The composition may optionally comprise one or more additional enzymes. The additional enzyme may be a lipolytic enzyme, particularly phospholipase, galactolipase and/or triacyl glycerol lipase activity, e.g. as described in WO1998026057, WO200032758, WO200200852 or WO2002066622.

Further, the additional enzyme may be an amylase, a cyclodextrin glucanotransferase, a protease or peptidase, in particular an exopeptidase, a transglutaminase, a phospholipase, a cellulase, a hemicellulase, a glycosyltransferase, a branching enzyme (1,4-alpha-glucan branching enzyme) or an oxidoreductase. The additional enzyme may be of mammalian, plant or microbial (bacterial, yeast or fungal) origin.

The amylase may be a maltogenic alpha-amylase (EC 3.2.1.133), e.g. from B. stearothermophilus, an alpha-amylase, e.g. from Bacillus, particularly B. licheniformis or B. amyloliquefaciens, a beta-amylase, e.g. from plant (e.g. soy bean) or from microbial sources (e.g. Bacillus), a glucoamylase, e.g. from A. niger, or a fungal alpha-amylase, e.g. from A. oryzae.

The hemicellulase may be a pentosanase, e.g. a xylanase which may be of microbial origin, e.g. derived from a bacterium or fungus, such as a strain of Aspergillus, in particular of A. aculeatus, A. niger, A. awamori, or A. tubigensis, from a strain of Trichoderma, e.g. T. reesei, or from a strain of Humicola, e.g. H. insolens.

The protease may be from Bacillus, e.g. B. amyloliquefaciens.

The oxidoreductase may be a glucose oxidase, a hexose oxidase, a lipoxidase, a peroxidase, or a laccase.

### Dough

The glucose dehydrogenase of the invention, e.g., in the form of the composition of the invention, may be added to a dough comprising flour. The flour may be derived from any cereal grain, including wheat, barley, rye, oat, corn, sorghum, rice and millet. In a preferred embodiment the flour is derived from wheat.

In addition the dough may comprise types of refined flour or starch as well as whole meal flour. Whole meal flour may be derived from grinding of cereal grains and is defined as flour comprising the components of the starchy endosperm, germ and bran in substantially the same relative proportions as they exist in the intact cereal grains. The production of whole meal flour may include temporary separation of the grains constituents for later recombination.

The dough of the invention may be fresh, frozen or par-baked.

The dough of the invention is normally a leavened dough or a dough to be subjected to leavening. The dough may be leavened in various ways, such as by adding chemical leavening agents, e.g., sodium bicarbonate or by adding a leaven (fermenting dough), but it is preferred to leaven the dough by adding a suitable yeast culture, such as a culture of Saccharomyces cerevisiae (baker's yeast), e.g. a commercially available strain of S. cerevisiae.

The dough may also comprise other conventional dough ingredients, e.g.: proteins, such as milk powder, gluten, and soy; eggs (either whole eggs, egg yolks or egg whites); an oxidant such as ascorbic acid, potassium bromate, potassium iodate, azodicarbonamide (ADA) or ammonium persulfate; an amino acid such as L-cysteine; a sugar; a salt such as sodium chloride, calcium acetate, sodium sulfate or calcium sulfate.

The dough may comprise fat (triglyceride) such as granulated fat or shortening, but the invention is particularly applicable to a dough where less than 1% by weight of fat (triglyceride) is added, and particularly to a dough which is made without addition of fat.

The dough may further comprise an emulsifier such as mono- or diglycerides, diacetyl tartaric acid esters of mono- or diglycerides, sugar esters of fatty acids, polyglycerol esters of fatty acids, lactic acid esters of monoglycerides, acetic acid esters of monoglycerides, polyoxyethylene stearates, or lysolecithin.

The amount of glucose dehydrogenase in the dough may be between 0.5-100 mg polypeptide per kg dry matter in the dough, in particular 0.5-50 mg polypeptide per kg dry matter, in particular 1-25 mg polypeptide per kg dry matter, in particular 1-15 mg polypeptide per kg dry matter in the dough, in particular 2-10 mg/kg.

### Edible Product

The dough may be used to prepare an edible product, such as a baked product, e.g. by leavening the dough and heating it, e.g. by baking or steaming. The product may be of a soft or a crisp character, either of a white, light or dark type. Examples are steamed or baked bread (in particular white, whole-meal or rye bread), typically in the form of loaves or rolls, pan bread, French baguette-type bread, pita bread, tortillas, cakes, pancakes, biscuits, cookies, pie crusts, crisp bread, steamed bread, pizza and the like.

The present invention is further described by the following examples that should not be construed as limiting the scope of the invention.

### Methods and Materials

### Enzymes

Pentopan Mono BG is a commercial enzyme composition comprising endo-1,4-xylanase. activity (2500 FXU-W/g).

Gluzyme Mono 10000 BG is a commercial enzyme composition comprising glucose oxidase activity (10000 GODU/g).

Fungamyl 2500 SG is a commercial enzyme composition comprising fungal alpha-amylase (2500 FAU-F/g).

Examples 2-4 were performed using glucose dehydrogenase isolated from the native host Glomerella cingulata. Examples 5-8 were performed using glucose dehydrogenase recombinantly produced in Aspergillus oryzae.

The commercial enzyme compositions are available from Novozymes NS.

### EXAMPLE 1

### Isolation of Glucose Dehydrogenase Encoding cDNA, and Expression of a Synthetic Codon Optimized Gene in Aspergillus oryzae

The gene was cloned from the strain Glomerella cingulata DSM 62728. Samples for nucleic acid isolation were taken from fresh shaking flask cultures grown on production medium to induce expression of glucose dehydrogenase. Mycelia were squeezed dry between filter paper and then frozen in liquid nitrogen and portions of approx. 100 mg were used for DNA extraction. Total RNA was isolated, cDNA synthesis performed and the resulting PCR product of ˜1.8 kb in length was sequenced to yield the coding DNA sequence shown in SEQ ID NO:1. Based on the native coding DNA sequence the synthetic gene codon optimized shown in SEQ ID NO:3 was designed for, and transformed into Aspergillus oryzae for expression of glucose dehydrogenase. SEQ ID NO:1 is 79.5% identical to SEQ ID NO:3; both encode the dehydrogenase shown in SEQ ID NO:2.

### EXAMPLE 2

### Electrophoretic Analysis

For SDS-PAGE, deglycosylation was performed under denaturing conditions. Glucose dehydrogenase was treated with PNGase F (New England Biolabs) according to the manufacturer's instructions. For isoelectric focusing, 0.1 mg of glucose dehydrogenase was deglycosylated under non-denaturating conditions with 1 μL Endo HF (New England Biolabs) and 1 μL α-mannosidase (from Jack bean, Sigma Aldrich) in 100 μL 50 mM sodium citrate buffer pH 5.5 containing 10 mM ZnCl₂for 48 h at 22° C.

SDS-PAGE was carried out in a Phast System using precast gels (PhastGel Gradient 8-25 and SDS buffer strips, GE Healthcare Biosciences) according to the manufacturer's modifications of the Laemmli procedure. Proteins and the Precision Plus Protein Dual Color standard (Bio-Rad) were visualized by Coomassie Brilliant Blue staining. For native PAGE different precast gels were used in the same horizontal electrophoresis system (PhastGel Gradient 8-25 and native buffer strips, GE Healthcare Biosciences) according to the manufacturer's recommendations together with the high molecular-weight native marker kit (GE Healthcare Biosciences). Isoelectric focusing (IEF) in the range of pH 3 to 10 was also performed in the Phast System using precast gels (PhastGel IEF 3-9, GE Healthcare Biosciences). The broad range pl marker protein kit (pH 3-10, Serva, Heidelberg, Germany) was used to determine the isoelectric point. The glucose dehydrogenase bands were visualized by active staining. For that purpose the gel was soaked in 3 mM dichlorophenolindophenol (DCPIP) solution, washed in 50 mM sodium citrate buffer, pH 5.0 and developed in a 2 mM glucose solution. A colorless band in a purple background appeared due to DCPIP reduction by glucose dehydrogenase. The position of this band was compared to the Coomassie Brilliant Blue stained pl standard. SDS-PAGE after deglycosylation under denaturing conditions with PNGase F resulted in a single, sharp band with an estimated molecular mass of 68 kDa. Isoelectric focusing showed that the glucose dehydrogenase had an acidic isoelectric point at pH 5.6.

### EXAMPLE 3

### Activity and Protein Measurements

Enzymatic activity of glucose may be spectrophotometrically assayed using dichlorophenolindophenol (DCPIP) (ε₅₂₀=6.9 mM⁻¹ cm⁻¹) as electron acceptor. The reaction is followed for 180 sec at 30° C. e.g., in a Lambda 35 UV/Vis spectrophotometer featuring a temperature controlled 8-cell changer (Perkin Elmer). The assay contain (final concentrations) 50 mM sodium acetate buffer, pH 5.5, 300 μM DCPIP and 100 mM D-glucose.

One unit of glucose dehydrogenase activity was defined as the amount of enzyme necessary for the reduction of 1 μmol glucose per min under the above assay conditions.

The protein concentration was determined by the method of Bradford using a prefabricated assay (Bio-Rad Laboratories) and bovine serum albumin as standard.

The glucose dehydrogenase activity of the polypeptide in SEQ ID NO:2 was determined to be approximately 840 U/mg enzyme protein.

### EXAMPLE 4

### Determination of the Reaction Product

A batch conversion (5 mL reaction volume) was carried out under continuous sparging and stirring at 30° C. The reaction mixture consisted of 50 mM D-glucose, 0.1 mM dichlorophenolindophenol (DCPIP), 3 U of laccase from Trametes pubescens (Galhaup et al 2001. Appl. Microbiol. Biotechnol. 56:225-232) and 3 U of glucose dehydrogenase in 100 mM citrate buffer, pH 5.0. The pH was measured but allowed to float freely. Samples were frequently taken and immediately denatured at 95° C. for 5 minutes. After removing the precipitate by centrifugation (10000×g, 10 min), diluted samples were analyzed by HPLC using a CarboPac PA1 carbohydrate analytical column with the dimensions 4×250 mm and a 4×50 mm guard column and the ED40 electrochemical detector (Dionex, Sunnyvale, Calif., USA).

Analysis was performed by developing a linear gradient from 0-50 mM sodium acetate in 100 mM sodium hydroxide solution at a flow rate of 0.5 mL min⁻¹ in 20 minutes. Five-point calibration curves for glucose, gluconic acid and glucuronic acid were established for a concentration range of 1-100 ppm.

To determine the reaction product of glucose dehydrogenase, a batch conversion of glucose by glucose dehydrogenase and an enzymatic regeneration system (Van Hecke et al 2009. Biotechnol. Bioeng. 102:1475-1482.) was performed. Glucose consumption was accompanied by a steady decrease of pH, indicating an acidic reaction product. HPLC analysis showed the formation of the C1 glucose oxidation product—gluconic acid—in an amount equivalent to that of the employed glucose. The enzymes ability to oxidize other carbohydrates at a 50 mM concentration was examined at pH 5.5 with DCIP as electron acceptor. A strong preference for glucose and the hemicellulose pentose xylose (31% relative activity compared to glucose) was observed. The relative conversion rates of the disaccharides cellobiose and maltose were very low (1.6 and 2.3%, respectively) and lactose was not converted at all. The conversion rates of the monosaccharides D-galactose, D-mannose and L-arabinose (0.2, 0.9 and 0.1% relative activity, respectively) were also very low.

### EXAMPLE 5

### Enzyme Characterization

Glucose dehydrogenase was characterized in terms of substrate specificity, pH profile and thermal stability. Substrate specificity was assessed with the carbohydrates: cellobiose solution (0.5 M), D(+)glucose solution (1 M), maltose solution (1 M), D(+)galactose solution (1 M) and D(+)xylose solution (1 M). The glucose dehydrogenase activity, in the presence of each carbohydrate substrate, was determined spectrophotometrically by reduction of DCPIP at 25° C.: 20 μL enzyme was added to a 0.5 cm microtiter plate compartment containing 100 μL Briton Robinson buffer (100 mM, pH 6.0), 20 μl carbohydrate solution, 40 μl H2O and 20 μl of DCPIP (7.5 mM), to a final volume of 200 μl. The change in absorbance at 520 nm was monitored for 90 seconds. The slope of the progress curve was used to calculate the initial rate of the reaction. The glucose dehydrogenase has the highest activity with D(+)glucose, and trace activity with cellobiose, maltose, D(+)galactose and D(+)xylose at the given concentrations. The relative activities are shown on table 1.

TABLE 1
Substrate specificity of glucose dehydrogenase

| Substrate | Relative activity |
|---|---|
| Cellobiose 50 mM | 0.02 |
| D(+)Glucose 100 mM | 1 |
| D(+)Xylose 100 mM | 0.06 |
| Maltose 100 mM | 0.05 |
| D(+)Galactose 100 mM | 0.03 |

The pH profile was assessed with the Britton-Robinson buffer pH 3.0-9.8. The glucose dehydrogenase activity at varying pHs was determined spectrophotometrically by reduction of DCPIP at 25° C.: 20 μL enzyme was added to a 0.5 cm microtiter plate compartment containing 100 μL 100 mM Briton Robinson buffer, 20 μl glucose solution (1 M), 40 μl H₂O and 20 μl of DCPIP (7.5 mM), to a final volume of 200 μl. The change in absorbance at 520 nm was monitored for 90 seconds. The slope of the progress curve was used to calculate the initial rate of the reaction. The pH profile of the glucose dehydrogenase is shown in table 2. The enzyme has an optimum at pH 6.

TABLE 2
pH profile of the glucose dehydrogenase

| pH | Relative Activity |
|---|---|
| 3.0 | 0.21 |
| 4.0 | 0.44 |
| 5.0 | 0.7 |
| 6.0 | 1 |
| 7.0 | 0.89 |
| 8.1 | 0.66 |
| 9.1 | 0.51 |
| 9.8 | 0.28 |

### EXAMPLE 6

### Determination of Melting Temperature of the Glucose Dehydrogenase

The thermostability of the glucose dehydrogenase was determined by Differential Scanning Calorimetry (DSC) using a VP-Capillary Differential Scanning Calorimeter (MicroCal Inc., Piscataway, N.J., USA). The thermal denaturation temperature, Td (° C.), was taken as the top of denaturation peak (major endothermic peak) in thermograms (Cp vs. T) obtained after heating variant enzyme solutions in buffer (K-Phosphate buffer pH 7.0) at a constant programmed heating rate of 200 K/hr.

Sample- and reference-solutions (approx. 0.2 ml) were loaded into the calorimeter (reference: buffer without enzyme) from storage conditions at 10° C. and thermally pre-equilibrated for 20 minutes at 20° C. prior to DSC scan from 20° C. to 110° C. Denaturation temperatures were determined at an accuracy of approximately +/−1° C. The DSC of the glucose dehydrogenase results was 54.4° C.

### EXAMPLE 7

### Baking of Pan Bread from Straight Dough

Pan bread was prepared from straight dough. Dough was prepared from the following ingredients:

| | | |
|---|---|---|
| Wheat flour | 2000 | g (100%)* |
| Water | 1152 | g (58%) |
| Yeast | 80 | g (4%) |
| Sugar | 30 | g (1.5%) |
| Salt | 30 | g (1.5%) |
| Ascorbic acid | 40 | mg (20 ppm) |
| Pentopan Mono | 72 | mg (36 ppm) |
| Fungamyl 2500 | 8 | mg (4 ppm) |
| SG | | |
| Gluzyme Mono | 0 or 16 | mg (0 or 8 ppm) |
| BG | | |
| Glucose | 0, 0.4, 1 or 4 | mg enzyme (0, 0.2, 0.5 and 2 mg/kg flour) |
| dehydrogenase | | |

*numbers in brackets show amount relative to flour weight (bakers percentage)

After mixing dough properties were evaluated (stickiness, softness, extensibility, elasticity and machine-ability) and the doughs were left to rest on the bench for 15 min. Dough properties were re-evaluated and doughs were left to rest for another 10 min. Subsequently, the different doughs were sheeted, moulded and 350 g dough was transferred to open pans (1230 mL). Dough was proofed for 55 min at 32° C./86% RH. After proofing doughs were baked for 230° C. for and 35 min. The baked bread was left to cool at room temperature for 2 hours. Specific bread volume was determined by rapeseed displacement and crumb structure was evaluated visually after the bread was cut open.

TABLE 3
Volume of pan bread prepared with glucose dehydrogenase
(DHG) or glucose oxidase (Gluzyme mono BG). Specific
volume is mean of four breads from each treatment.

| | Specific volume | Specific volume |
|---|---|---|
| Sample | (g/mL) | in % of control |
| Control | 4.29 | 100 |
| Gluzyme mono BG | 4.28 | 100 |
| 0.2 mg DHG/kg flour | 4.15 | 97 |
| 0.5 mg DHG/kg flour | 4.38 | 102 |
| 2.0 mg DHG/kg flour | 4.22 | 98 |

### Dough Handling Properties:

The addition of glucose dehydrogenase resulted in a lower stickiness, shorter extensibility, lower softness, higher elasticity and overall dryer dough compared to the control. The effect of glucose dehydrogenase and glucose oxidase was found to be very similar.

### Bread Volume

No significant differences in bread volume were observed for the different bread samples.

### Crumb Structure:

Addition of glucose dehydrogenase resulted in a finer and more uniform crumb structure comparable to the crumb structure of the control as well as of the glucose oxidase treatment. The open and less uniform crumb structure (less desirable) often experienced by the use of glucose oxidase and also present in this trial was not observed when glucose dehydrogenase was added.

### Overall Performance:

Addition of glucose dehydrogenase contributed positively to dough stability and final bread quality. The effect of glucose dehydrogenase was considered to be similar or better than glucose oxidase on all parameters tested.

### Example 8

### Baking of French Baguettes

French baguettes were prepared from straight dough. Dough was prepared from the following ingredients:

| | | |
|---|---|---|
| Wheat flour | 2000 | g (100%)* |
| Water | 1133 | g (57%) |
| Yeast | 50 | g (2.5%) |
| Salt | 44 | g (2.2%) |
| Ascorbic acid | 60 | mg (30 ppm) |
| Pentopan Mono | 72 | mg (36 ppm) |
| Fungamyl 2500 | 8 | mg (4 ppm) |
| SG | | |
| Gluzyme Mono | 0 or 16 | mg (0 or 8 ppm) |
| BG | | |
| Glucose | 0, 0.02, 0.04, 0.08 or 0.16 | mg (0-0.08 mg/kg flour) |
| dehydrogenase | | |

*numbers in brackets show amount relative to flour weight (bakers percentage)

After mixing and resting on the bench for 15 min, the doughs were evaluated (stickiness, softness, extensibility, elasticity and machine-ability). Subsequently, the doughs were proofed for 120 min at 30° C./80% RH. After proofing the doughs were baked for 230° C. for 22 min. The baked bread was left to cool at room temperature for 2 hours. Specific bread volume was determined by rapeseed displacement. Bloom was evaluated visually. Crumb structure was evaluated visually after the bread was cut open.

TABLE 4
Volume of French baguette prepared with glucose dehydrogenase
(DHG) or glucose oxidase (Gluzyme mono BG). Specific volume
is mean of three breads from each treatment.

| | Specific volume | Specific volume |
|---|---|---|
| Sample | (g/mL) | in % of control |
| Control | 19.80 | 100 |
| Gluzyme mono BG | 21.83 | 110 |
| 0.01 mg DHG/kg flour | 20.90 | 106 |
| 0.02 mg DHG/kg flour | 21.07 | 106 |
| 0.04 mg DHG/kg flour | 20.98 | 106 |
| 0.08 mg DHG/kg flour | 19.63 | 99 |

### Dough Handling Properties (Machine-Ability)

The addition of glucose oxidase resulted in shorter extensibility and higher elasticity compared to the control and glucose dehydrogenase containing dough. No significant difference between control and treatment with glucose dehydrogenase were observed.

### Bread Volume

Bread volume was found to increase in both dehydrogenase and glucose oxidase treatments as shown in table XX.

### Bloom

Bloom in baguettes containing glucose dehydrogenase and glucose oxidase was found to be substantially wider and more open compared to the bloom obtained for the control.

### Crumb Structure

No significant differences in crumb structure between treatments were observed in this trial using low amounts of GDH.

The invention described and claimed herein is not to be limited in scope by the specific aspects herein disclosed, since these aspects are intended as illustrations of several aspects of the invention. Any equivalent aspects are intended to be within the scope of this invention. Indeed, various modifications of the invention in addition to those shown and described herein will become apparent to those skilled in the art from the foregoing description. Such modifications are also intended to fall within the scope of the appended claims. In the case of conflict, the present disclosure including definitions will control.

1. An isolated polypeptide having glucose dehydrogenase activity, selected from the group consisting of:
(a) a polypeptide having at least 60% sequence identity to the mature polypeptide of SEQ ID NO: 2;
(b) a polypeptide encoded by a polynucleotide that hybridizes under low stringency conditions, medium stringency conditions, medium-high stringency conditions, high stringency conditions, or very high stringency conditions with (i) the mature polypeptide coding sequence of SEQ ID NO: 1 or SEQ ID NO: 3, or (ii) the full-length complement of (i);
(c) a polypeptide encoded by a polynucleotide having at least 60% sequence identity to the mature polypeptide coding sequence of SEQ ID NO: 1 or SEQ ID NO: 3;
(d) a variant of the mature polypeptide of SEQ ID NO: 2 comprising a substitution, deletion, and/or insertion at one or more positions; and
(e) a fragment of the polypeptide of (a), (b), (c), or (d) that has glucose dehydrogenase activity.

2. The polypeptide of claim 1, comprising an amino acid sequence having at least 80% identity to the mature polypeptide of SEQ ID NO: 2.

3. The polypeptide of claim 1, comprising an amino acid sequence having at least 85% identity to the mature polypeptide of SEQ ID NO: 2.

4. The polypeptide of claim 1, comprising an amino acid sequence having at least 90% identity to the mature polypeptide of SEQ ID NO: 2.

5. The polypeptide of claim 1, comprising an amino acid sequence having at least 95% identity to the mature polypeptide of SEQ ID NO: 2.

6. The polypeptide of claim 1, comprising or consisting of the amino acid sequence of SEQ ID NO: 2, the mature polypeptide of SEQ ID NO: 2 or a fragment thereof.

7. The polypeptide of claim 1, wherein the mature polypeptide is amino acids 1 to 586 of SEQ ID NO: 2.

8. The polypeptide of claim 1, which is a variant of the mature polypeptide of SEQ ID NO: 2 comprising a substitution, deletion, and/or insertion at one or more positions.

9. The polypeptide of claim 1, which is a fragment of SEQ ID NO: 2, wherein the fragment has glucose dehydrogenase activity.

10. The polypeptide of claim 1, which is encoded by a polynucleotide that hybridizes under low stringency conditions, low-medium stringency conditions, medium stringency conditions, medium-high stringency conditions, high stringency conditions, or very high stringency conditions with (i) the mature polypeptide coding sequence of SEQ ID NO: 1 or SEQ ID NO: 3, or (ii) the full-length complement of (i).

11. A composition comprising the polypeptide of claim 1.

12. The composition of claim 11, further comprising a carrier selected from a salt, a sugar, a sugar alcohol, starch, rice, corn grits, or soy.

13. The composition of claim 11, in the form of a granulate or agglomerated powder.

14. The composition of claim 11, wherein more than 95% (by weight) of the granulate or agglomerated powder has a particle size between 25 and 500 μm.

15. The composition of claim 11 which is a dough.

16. An isolated polynucleotide encoding the polypeptide of claim 1.

17. A nucleic acid construct or expression vector comprising the polynucleotide of claim 16 operably linked to one or more control sequences that direct the production of the polypeptide in an expression host.

18. A recombinant host cell comprising the polynucleotide of claim 16 operably linked to one or more control sequences that direct the production of the polypeptide.

19. A method of producing the polypeptide of claim 1, comprising:
(a) cultivating a cell, which in its wild-type form produces the polypeptide, under conditions conducive for production of the polypeptide; and
(b) recovering the polypeptide.

20. A method of producing a polypeptide having glucose dehydrogenase activity, comprising:
(a) cultivating the host cell of claim 18 under conditions conducive for production of the polypeptide; and
(b) recovering the polypeptide.


SEQUENCE LISTING (4 sequences)

SEQ ID NO: 1 - DNA, 1803 bp, Glomerella cingulata
  CDS (1)..(1800)
  sig_peptide (1)..(42)
  mat_peptide (43)..(1800)
atgaaaaacttgattcccttgtcgctcctcgccacgacagtcgcagcccgacccggatcggcacctcgcgaccaggcagcagccacagcatacgactacattgtcattggaggcggaacgtcgggactcgtggtggccaaccgactctccgaggatgcctccgtctccgtgttggtgattgaggcaggtgattccgtgctcaacaacgcaaacgtcacaaacgccaacggttacggcctcgcgttcggcacggacattgattacgcctatcagaccacagcacagacttacgcaaacaacgcgtcgacaaccttgcgagcagccaaggccctcggaggcacgtccactatcaacggaatggcctacacacgagcggaagcctcgcagatcgatgcctgggagacggtcggcaacgagggttggaactgggatgccctcctcccctattacctcaagtcggagaccttccaggcacccgacgcagaacgctcgattaagggccatatctcgtatgagtcggacgtgcacggtcatgatggtccgctctacaccgcatacgcctatggatcgaccaacgactcgtaccccacctcgctcaacgcaacctaccaggcgctcaacgtcccctggaaagaggatatcgcaggcggttcgatggtgggcttcgcgtcgtatcccaagacactcaaccaggacttgaacatccgatgggatgcagcgagggcgtactacttcccctacgagaacaggaccaacctcaaggtcgtcctcaacacaaccgcaaagaaactcacatgggcgtccgcaaccaacggcaccgatgccacagcgtcgggagtggagatcactgcagccgatggcaccacttccgtcgtcaccgcaaacaaagaggtgattatctccgcaggagcgctcgtgtcgcccttgctcttggagctctcgggagtgggcaaccctgcatggctctcccagtatggaatcgagacagtcgtcgagctccctactgtcggtgaaaacttgcaggaccagatcaacaacgagttgatctattcccctccgacaaacttcacatcgacgtacgattccggagtcggagcattcgtggcctacccgtcggcgtcgcacgtcttcggaaccaacgaatcgtccgcctccgaagagttgaagtcgcagctcactgcctacgcagacaccgtggcgatcgccaacggcaacgtcacaaaggcgtcggatctcctcgatttcttccagctccagtatgacctcatcttcaaagatcaggtgcctttcgcagaggtcttgatctatatcgccaaaggctcctggggagcagagtactggggactcctccctttctcgcgaggctccattcatatctcccaggcaaactcgacagcaggtgcattgatcaaccccaactacttcatgctcgattacgatgtcgaattgcaggtggccacggccaaattcatccggtcggtgttcggcacaggaccgttcgcgtccgtggcaggcactgagacaacacctggcttcgatgtgattcccgcagatgcggatgaggcgacctggaaatcctgggcgaccaaggagtaccggtccaacttccatcctgtggcaactgcggcaatgctccctaaagagaagggaggcgtcgtcgacgcgcagctcaaggtgtatggcacgaccaacgtgcgggtggtggatgcctcggtgctccccttccaggtgtgtggccacttggtgtccacgctctacgccgtggccgagaaggcgtcggacttgatcaaagcagcagcgtga

SEQ ID NO: 2 - protein, 600 aa, Glomerella cingulata
MKNLIPLSLLATTVAARPGSAPRDQAAATAYDYIVIGGGTSGLVVANRLSEDASVSVLVIEAGDSVLNNANVTNANGYGLAFGTDIDYAYQTTAQTYANNASTTLRAAKALGGTSTINGMAYTRAEASQIDAWETVGNEGWNWDALLPYYLKSETFQAPDAERSIKGHISYESDVHGHDGPLYTAYAYGSTNDSYPTSLNATYQALNVPWKEDIAGGSMVGFASYPKTLNQDLNIRWDAARAYYFPYENRTNLKVVLNTTAKKLTWASATNGTDATASGVEITAADGTTSVVTANKEVIISAGALVSPLLLELSGVGNPAWLSQYGIETVVELPTVGENLQDQINNELIYSPPTNFTSTYDSGVGAFVAYPSASHVFGTNESSASEELKSQLTAYADTVAIANGNVTKASDLLDFFQLQYDLIFKDQVPFAEVLIYIAKGSWGAEYWGLLPFSRGSIHISQANSTAGALINPNYFMLDYDVELQVATAKFIRSVFGTGPFASVAGTETTPGFDVIPADADEATWKSWATKEYRSNFHPVATAAMLPKEKGGVVDAQLKVYGTTNVRVVDASVLPFQVCGHLVSTLYAVAEKASDLIKAAA

SEQ ID NO: 3 - DNA, 1803 bp, Artificial
  Codon optimized DNA
atgaagaacctcattcctctttccctccttgccactaccgtggccgctcggcccgggtctgcccccagggaccaggctgctgccacggcatacgactatattgtcattggtggcggtaccagcggtctggtcgtcgcaaaccgcctgtctgaagacgcgtctgtctccgtgcttgtcatcgaggccggtgactccgtcttgaacaacgccaatgtcactaatgccaacgggtacggcttggccttcggaaccgatatcgactatgcctaccagacgacggcacagacatacgccaacaatgcttccacgaccctgagggccgccaaggcccttggtggaaccagcaccatcaacgggatggcttacacacgggctgaggcttcgcaaatcgacgcatgggagactgttggaaacgagggctggaactgggacgcactgctcccatactacctgaagagtgagaccttccaggcaccggacgctgagcgttcgatcaagggtcacataagctacgaatccgatgtccacggtcacgatggcccgctctacacggcctatgcttacggctcgaccaacgactcgtaccctactagtctgaatgcgacataccaggctcttaacgtgccatggaaggaagacattgctggtggtagcatggtcgggttcgcgtcctaccccaagactctcaaccaggacctcaacatccgttgggacgcagccagggcatactacttcccctacgagaaccgcacgaacctcaaggtggttctcaacacgaccgccaagaagctcacctgggccagcgccaccaacggcaccgatgctaccgccagcggcgtcgagatcaccgccgccgacggcaccacctcagtcgtcacggctaacaaggaggtcatcatctccgccggcgccctcgtctcccccctgctcctcgagctctccggagtgggcaaccccgcgtggctgtcccaatacggtatcgagactgttgtcgaactccccaccgtcggcgagaacctgcaggaccagatcaacaacgagctgatctacagcccgcccaccaacttcacctccacctatgactctggcgtcggggcgttcgtcgcctacccctccgcgtctcatgtcttcggcaccaacgagtcaagtgcctccgaggagctcaagtcccagctaaccgcctacgccgacaccgtagccatcgccaatggcaacgtcaccaaggccagcgacctgctcgacttcttccagctccagtacgacctgatcttcaaggaccaggtgccgttcgccgaggtcctcatctacatcgccaagggctcctggggtgccgagtactggggactcctgcccttctcccgcggctccatccacatcagccaggccaactcgaccgccggcgccctcatcaaccccaactacttcatgctggactacgatgtcgagctgcaggtcgccaccgccaagttcatccgcagcgtctttggcaccgggccgttcgcgtccgtcgcgggcacggagaccacccccggcttcgatgtcatccctgccgatgcggacgaagccacctggaagtcttgggccaccaaggaataccgctccaacttccaccccgtcgcgaccgccgccatgttgcccaaggagaagggcggtgttgtggatgcccagctcaaggtctacggaactaccaatgtccgcgtggttgacgcgtcggtccttcccttccaggtctgcggacacttggtcagcacgctctatgctgttgccgagaaggcgtctgatctgatcaaggctgctgcttga

SEQ ID NO: 4 - protein, 600 aa, Artificial; identical to SEQ ID NO: 2
  Synthetic Construct